(19) United States
(12) Patent Application Publication
(10) Pub. No.: US 2025/0302408 A1
(43) Pub. Date: Oct. 2, 2025

(54) RADIOLOGICAL IMAGING METHOD WITH A MULTI-ENERGY SCAN IMAGE

(71) Applicant: EOS IMAGING, PARIS (FR)

(72) Inventors: Jérôme BEUCHER, ESBLY (FR); Pascal DESAUTE, PARIS (FR); Pierre MORICHAU-BEAUCHANT, PARIS (FR); Aymeric RESHEF, PARIS (FR)

(73) Assignee: EOS IMAGING, PARIS (FR)

(21) Appl. No.: 18/866,671

(22) PCT Filed: May 18, 2022
(86) PCT No.: PCT/EP2022/063405

§ 371 Date: Nov. 18, 2024

(51) Int. Cl. A61B 6/40; A61B 6/00; A61B 6/42

(52) U.S. Cl. CPC A61B 6/4014; A61B 6/4241; A61B 6/482; A61B 6/544

(57) ABSTRACT

A radiological imaging method including 2 radiation detectors respectively associated with 2 radiations sources. The method includes an operating mode making, combining, and processing frontal and lateral multi-energy scout views, so as to evaluate a patient's bone thickness, soft tissue thickness, and specific bone localization at different imaging positions along the vertical scanning direction so that in a single vertical scanning: a frontal multi-energy image made, wherein a frontal radiation detector provides a first frontal image of low energy, a second frontal image of high energy, and a combined frontal image from the combination of these frontal images; and a lateral multi-energy image is made, wherein a lateral radiation detector provides a first lateral image of low energy, a second lateral image of high energy, and a combined lateral image from the combination of these lateral images.

### FIELD OF THE INVENTION

The invention relates to the technical field of radiological imaging method and of radiological apparatus for performing this radiological method.

### BACKGROUND OF THE INVENTION

Different types of radiological images can be done, among which:
radiological scan image dedicated to a total (detailed and complete) view of a patient, or of one or more patient organ(s), or of a part of a patient organ, which is used for diagnosis by practitioner,
radiological scan image dedicated to bone density distribution within a patient, or within one or more patient organ(s), or within a part of a patient organ, which is used for bone density evaluation by practitioner.

Radiological image is preferably X-ray image.

In order to improve accuracy of diagnosis and/or bone density evaluation, in a first step, a scout view is performed, and then using information extracted from this scout view to adapt imaging parameters, one or more scan image(s) is or are performed which is or are then used by the practitioner, either for diagnosis or for bone density evaluation.

Scout view and scan image are performed by vertical scanning along the height of a standing patient, of a frontal image taking-line including a frontal radiation source and a frontal radiation detector and/or of a lateral image taking-line including a lateral radiation source and a lateral radiation detector. Scout view is performed with about 10 times less radiation dose or even less, as compared to scan image.

According to a first prior art, when performing a mono-energy scout view followed by a mono-energy scan image, imaging parameters are adapted so as to allow to get a good quality diagnosis image but which could not give good result for bone density evaluation, i.e. from which diagnosis image no good result for bone density evaluation can be derived. Then, if bone density evaluation is also needed, not only a new scan image with different imaging parameters should be done, but also this new scan image cannot topologically correspond exactly to the former scan image, because the standing patient would have moved, at least a little, in between.

According to a second prior art, performing a multi-energy scan image, imaging parameters are adapted so as to allow to get a good quality bone density image but which could not give good result for diagnosis, i.e. from which bone density image no good result for diagnosis image. Then, if diagnosis is also needed, not only a new scan image with different imaging parameters should be done, but also this new scan image cannot topologically correspond exactly to the former scan image, because the standing patient would have moved, at least a little, in between.

### SUMMARY OF THE INVENTION

The object of the present invention is to alleviate at least partly the above mentioned drawbacks.

More particularly, the invention aims at providing for a scan image which can be used for diagnosis with good result, but from which, at the same time, partial images can be extracted and further combined so as to give good results for bone density evaluation too. A combination of both partial images will lead to accurate evaluation of bone density. So, good diagnosis and good bone density evaluation can be then both derived from the same scan image. Besides, since diagnosis and good bone density evaluation are both derived from the same scan image, there will be an exact topological correspondence between diagnosis and bone density evaluation, because the standing patient is exactly in the same position for both.

Therefore, in order to be useful, with good quality, both for diagnosis and for bone density evaluation, the radiological method uses:
first, a scout view which is either mono-energy or multi-energy, this scout view being only mono-energy in prior art,
second, extract information from this scout view, which information is used to then perform a multi-energy scan image, this scan image being only mono-energy in prior art.

The multi-energy scan image will give even better results if performed after a multi-energy scout view rather than after a mono-energy scout view.

The multi-energy scan image can be either a frontal image, or a lateral image, or include both frontal and lateral images.

A first object of the invention deals with a frontal multi-energy scan image, performed after a frontal mono-energy scout view.

This first object is achieved with a radiological imaging method comprising:
2 radiation sources with imaging directions orthogonal to each other, one frontal radiation source and one lateral radiation source, sliding vertically so as to perform vertical scanning of a standing patient along a vertical scanning direction,
2 radiation detectors which are respectively associated with said 2 radiations sources, one frontal radiation detector and one lateral radiation detector, sliding vertically so as to perform vertical scanning of a standing patient along said vertical scanning direction, at least said frontal radiation detector being a multi-energy counting detector,

wherein said radiological method comprises at least one operating mode in which:
a frontal mono-energy scout view is made by performing a preliminary vertical scanning of a standing patient along said vertical scanning direction by said frontal radiation source and by said frontal radiation detector,
said frontal scout view is processed to identify a patient thickness and a specific bone(s) localization at different positions along said vertical scanning direction within said frontal scout view,
a driving current intensity of at least said frontal radiation source is modulated along said vertical scanning direction, depending on identified patient thickness and on said identified specific bone(s) localization at different positions along said vertical scanning direction,
so that a frontal multi-energy image is made by performing a single vertical scanning of a standing patient along said vertical scanning direction by said frontal radiation source and by said frontal radiation detector,
   with a driving current intensity modulation of said frontal radiation source, with no voltage intensity modulation of said frontal radiation source, depending on said patient thickness and depending on said identified specific bone(s) localization at different positions along said vertical scanning direction, which is performed automatically, so as to improve a compromise between:
      the global radiation dose received by a patient during said vertical scanning,
      and the local image contrasts of said identified specific bone(s) localization at different imaging positions along said vertical scanning direction, for, at least one or more, or preferably all, frontal images given by said frontal radiation detector,
so that said frontal radiation detector gives at least, after said single vertical scanning:
   a first frontal image corresponding to a first portion of energy which is received by said frontal radiation detector and which is below a first given energy threshold, called low energy frontal image,
   a second frontal image corresponding to a second portion of energy which is received by said frontal radiation detector and which is above a second given energy threshold, called high energy frontal image,
   at least a combined frontal image corresponding to a combination of said first frontal image and said second frontal image.

A second object of the invention deals with a lateral multi-energy scan image, performed after a lateral mono-energy scout view.

This second object is achieved with a radiological imaging method comprising:
2 radiation sources with imaging directions orthogonal to each other, one frontal radiation source and one lateral radiation source, sliding vertically so as to perform vertical scanning of a standing patient along a vertical scanning direction,
2 radiation detectors which are respectively associated with said 2 radiations sources, one frontal radiation detector and one lateral radiation detector, sliding vertically so as to perform vertical scanning of a standing patient along said vertical scanning direction, at least said lateral radiation detector being a multi-energy counting detector,

wherein said radiological method comprises at least one operating mode in which:
a lateral mono-energy scout view is made by performing a preliminary vertical scanning of a standing patient along said vertical scanning direction by said lateral radiation source and by said lateral radiation detector,
said lateral scout view is processed to identify a patient thickness and a specific bone(s) localization at different positions along said vertical scanning direction within said lateral scout view,
a driving current intensity of at least said lateral radiation source is modulated along said vertical scanning direction, depending on identified patient thickness and on said identified specific bone(s) localization at different positions along said vertical scanning direction,
so that a lateral multi-energy image is made by performing a single vertical scanning of a standing patient along said vertical scanning direction by said lateral radiation source and by said lateral radiation detector,
   with a driving current intensity modulation of said lateral radiation source, with no voltage intensity modulation of said lateral radiation source, depending on said patient thickness and depending on said identified specific bone(s) localization at different positions along said vertical scanning direction, which is performed automatically, so as to improve a compromise between:
      the global radiation dose received by a patient during said vertical scanning,
      and the local image contrasts of said identified specific bone(s) localization at different imaging positions along said vertical scanning direction, for, at least one or more, or preferably all, lateral images given by said lateral radiation detector,
so that said lateral radiation detector gives at least, after said single vertical scanning:
   a first lateral image corresponding to a first portion of energy which is received by said lateral radiation detector and which is below a first given energy threshold, called low energy lateral image,
   a second lateral image corresponding to a second portion of energy which is received by said lateral radiation detector and which is above a second given energy threshold, called high energy lateral image,
   at least a combined lateral image corresponding to a combination of said first lateral image and said second lateral image.

A third object of the invention deals with both frontal and lateral multi-energy scan images, performed after both frontal and lateral mono-energy scout views.

This third object is achieved with a radiological imaging method comprising:
2 radiation sources with imaging directions orthogonal to each other, one frontal radiation source and one lateral radiation source, sliding vertically so as to perform vertical scanning of a standing patient along a vertical scanning direction,
2 radiation detectors which are respectively associated with said 2 radiations sources, one frontal radiation detector and one lateral radiation detector, sliding vertically so as to perform vertical scanning of a standing patient along said vertical scanning direction, said 2 radiation detectors being respectively 2 multi-energy counting detectors,

wherein said radiological method comprises at least one operating mode in which:
frontal and lateral mono-energy scout views are made by performing a preliminary vertical scanning of a standing patient along said vertical scanning direction by said frontal and lateral radiation sources and by said frontal and lateral radiation detectors,
said frontal and lateral scout views are processed to identify a patient thickness and a specific bone(s) localization at different positions along said vertical scanning direction within said frontal and lateral scout views,
driving current intensities of both said frontal and lateral radiation sources are modulated along said vertical scanning direction, depending on identified patient thickness and on said identified specific bone(s) localization at different positions along said vertical scanning direction,
so that a frontal multi-energy image is made by performing a single vertical scanning of a standing patient along said vertical scanning direction by said frontal radiation source and by said frontal radiation detector,
   with a driving current intensity modulation of said frontal radiation source, with no voltage intensity modulation of said frontal radiation source, depending on said patient thickness and depending on said identified specific bone(s) localization at different positions along said vertical scanning direction, which is performed automatically, so as to improve a compromise between:
      the global radiation dose received by a patient during said vertical scanning,
      and the local image contrasts of said identified specific bone(s) localization at different imaging positions along said vertical scanning direction, for, at least one or more, or preferably all, frontal images given by said frontal radiation detector,
so that said frontal radiation detector gives at least, after said single vertical scanning:
   a first frontal image corresponding to a first portion of energy which is received by said frontal radiation detector and which is below a first given energy threshold, called low energy frontal image,
   a second frontal image corresponding to a second portion of energy which is received by said frontal radiation detector and which is above a second given energy threshold, called high energy frontal image,
   at least a combined frontal image corresponding to a combination of said first frontal image and said second frontal image,
and so that a lateral multi-energy image is made by performing a vertical scanning of a standing patient along said vertical scanning direction by said lateral radiation source and by said lateral radiation detector,
   with a driving current intensity modulation of said lateral radiation source, with no voltage intensity modulation of said lateral radiation source, depending on said patient thickness and depending on said identified specific bone(s) localization at different positions along said vertical scanning direction, which is performed automatically, so as to improve a compromise between:
      the global radiation dose received by a patient during said vertical scanning,
      and the local image contrasts of said identified specific bone(s) localization at different imaging positions along said vertical scanning direction, for, at least one or more, or preferably all, lateral images given by said lateral radiation detector,
so that said lateral radiation detector gives at least:
   a first lateral image corresponding to a first portion of energy which is received by said lateral radiation detector and which is below a first given energy threshold, called low energy lateral image,
   a second lateral image corresponding to a second portion of energy which is received by said lateral radiation detector and which is above a second given energy threshold, called high energy lateral image,
   at least a combined lateral image corresponding to a combination of said first lateral image and said second lateral image,
both frontal and lateral multi-energy images being made during same vertical scanning.

A fourth object of the invention deals with a frontal multi-energy scan image, performed after a frontal multi-energy scout view.

This fourth object is achieved with a radiological imaging method comprising:
2 radiation sources with imaging directions orthogonal to each other, one frontal radiation source and one lateral radiation source, sliding vertically so as to perform vertical scanning of a standing patient along a vertical scanning direction,
2 radiation detectors which are respectively associated with said 2 radiations sources, one frontal radiation detector and one lateral radiation detector, sliding vertically so as to perform vertical scanning of a standing patient along said vertical scanning direction, said 2 radiation detectors being respectively 2 multi-energy counting detectors,

wherein said radiological method comprises at least one operating mode in which:
a frontal multi-energy scout view is made by performing a preliminary vertical scanning of a standing patient along said vertical scanning direction by said frontal radiation source and by said frontal radiation detector, so that said frontal radiation detector give at least:
   a first frontal scout view corresponding to a first portion of energy which is received by said frontal radiation detector and which is below a first given energy threshold, called low energy frontal scout view,
   a second frontal scout view corresponding to a second portion of energy which is received by said frontal radiation detector and which is above a second given energy threshold, called high energy frontal scout view,
said first frontal scout view and said second frontal scout view are combined and processed so as to evaluate:
   at least a patient bone thickness,
   at least a patient soft tissue thickness,
   a patient specific bone localization at different imaging positions along said vertical scanning direction,
driving current intensity of said frontal radiation source is modulated along said vertical scanning direction, depending on said patient bone thickness, on said patient soft tissue thickness, and on said identified specific bone(s) localization at different positions along said vertical scanning direction,
so that a frontal multi-energy image is made by performing a single vertical scanning of a standing patient along said vertical scanning direction by said frontal radiation source and by said frontal radiation detector,
   with a driving current intensity modulation of said frontal radiation source, with no voltage intensity modulation of said frontal radiation source, depending on said patient bone thickness, on said patient soft tissue thickness, and depending on said identified specific bone(s) localization at different positions along said vertical scanning direction, which is performed automatically, so as to improve a compromise between:
      the global radiation dose received by a patient during said vertical scanning, and the local image contrasts of said identified specific bone(s) localization at different imaging positions along said vertical scanning direction, for, at least one or more, or preferably all, frontal images given by said frontal radiation detector,
so that said frontal radiation detector gives at least, after said single vertical scanning:
   a first frontal image corresponding to a first portion of energy which is received by said frontal radiation detector and which is below a first given energy threshold, called low energy frontal image,
   a second frontal image corresponding to a second portion of energy which is received by said frontal radiation detector and which is above a second given energy threshold, called high energy frontal image,
   at least a combined frontal image corresponding to a combination of said first frontal image and said second frontal image.

A fifth object of the invention deals with a lateral multi-energy scan image, performed after a lateral multi-energy scout view.

This fifth object is achieved with a radiological imaging method comprising:
2 radiation sources with imaging directions orthogonal to each other, one frontal radiation source and one lateral radiation source, sliding vertically so as to perform vertical scanning of a standing patient along a vertical scanning direction,
2 radiation detectors which are respectively associated with said 2 radiations sources, one frontal radiation detector and one lateral radiation detector, sliding vertically so as to perform vertical scanning of a standing patient along said vertical scanning direction, said 2 radiation detectors being respectively 2 multi-energy counting detectors,

wherein said radiological method comprises at least one operating mode in which:
a lateral multi-energy scout view is made by performing a preliminary vertical scanning of a standing patient along said vertical scanning direction by said lateral radiation source and by said lateral radiation detector, so that said lateral radiation detector give at least:
   a first lateral scout view corresponding to a first portion of energy which is received by said lateral radiation detector and which is below a first given energy threshold, called low energy lateral scout view,
   a second lateral scout view corresponding to a second portion of energy which is received by said lateral radiation detector and which is above a second given energy threshold, called high energy lateral scout view,
said first lateral scout view and said second lateral scout view are combined and processed so as to evaluate:
   at least a patient bone thickness,
   at least a patient soft tissue thickness,
   a patient specific bone localization at different imaging positions along said vertical scanning direction,
driving current intensity of said lateral radiation source is modulated along said vertical scanning direction, depending on said patient bone thickness, on said patient soft tissue thickness, and on said identified specific bone(s) localization at different positions along said vertical scanning direction,
so that a lateral multi-energy image is made by performing a single vertical scanning of a standing patient along said vertical scanning direction by said lateral radiation source and by said lateral radiation detector,
   with a driving current intensity modulation of said lateral radiation source, with no voltage intensity modulation of said lateral radiation source, depending on said patient bone thickness, on said patient soft tissue thickness, and depending on said identified specific bone(s) localization at different positions along said vertical scanning direction, which is performed automatically, so as to improve a compromise between:
      the global radiation dose received by a patient during said vertical scanning,
      and the local image contrasts of said identified specific bone(s) localization at different imaging positions along said vertical scanning direction, for, at least one or more, or preferably all, lateral images given by said lateral radiation detector,
so that said lateral radiation detector gives at least, after said single vertical scanning:
   a first lateral image corresponding to a first portion of energy which is received by said lateral radiation detector and which is below a first given energy threshold, called low energy lateral image,
   a second lateral image corresponding to a second portion of energy which is received by said lateral radiation detector and which is above a second given energy threshold, called high energy lateral image,
   at least a combined lateral image corresponding to a combination of said first lateral image and said second lateral image.

A sixth object of the invention deals with both frontal and lateral multi-energy scan images, performed after both frontal and lateral multi-energy scout views.

This sixth object is achieved with a radiological imaging method comprising:
2 radiation sources with imaging directions orthogonal to each other, one frontal radiation source and one lateral radiation source, sliding vertically so as to perform vertical scanning of a standing patient along a vertical scanning direction,
2 radiation detectors which are respectively associated with said 2 radiations sources, one frontal radiation detector and one lateral radiation detector, sliding vertically so as to perform vertical scanning of a standing patient along said vertical scanning direction, said 2 radiation detectors being respectively 2 multi-energy counting detectors,

wherein said radiological method comprises at least one operating mode in which:
frontal and lateral multi-energy scout views are made by performing a preliminary vertical scanning of a standing patient along said vertical scanning direction by said frontal and lateral radiation sources and by said frontal and lateral radiation detectors, so that said frontal and lateral radiation detectors give at least:
   a first frontal scout view corresponding to a first portion of energy which is received by said frontal radiation detector and which is below a first given energy threshold, called low energy frontal scout view,
   a second frontal scout view corresponding to a second portion of energy which is received by said frontal radiation detector and which is above a second given energy threshold, called high energy frontal scout view,
   a first lateral scout view corresponding to a first portion of energy which is received by said lateral radiation detector and which is below a first given energy threshold, called low energy lateral scout view,
   a second lateral scout view corresponding to a second portion of energy which is received by said lateral radiation detector and which is above a second given energy threshold, called high energy lateral scout view,
said first frontal and lateral scout views and said second frontal and lateral scout views are combined and processed so as to evaluate:
   at least a patient bone thickness,
   at least a patient soft tissue thickness,
   a patient specific bone localization at different imaging positions along said vertical scanning direction,
driving current intensities of both said frontal and lateral radiation sources are modulated along said vertical scanning direction, depending on said patient bone thickness, on said patient soft tissue thickness, and on said identified specific bone(s) localization at different positions along said vertical scanning direction,
so that a frontal multi-energy image is made by performing a single vertical scanning of a standing patient along said vertical scanning direction by said frontal radiation source and by said frontal radiation detector,
   with a driving current intensity modulation of said frontal radiation source, with no voltage intensity modulation of said frontal radiation source, depending on said patient bone thickness, on said patient soft tissue thickness, and depending on said identified specific bone(s) localization at different positions along said vertical scanning direction, which is performed automatically, so as to improve a compromise between:
      the global radiation dose received by a patient during said vertical scanning,
      and the local image contrasts of said identified specific bone(s) localization at different imaging positions along said vertical scanning direction, for, at least one or more, or preferably all, frontal images given by said frontal radiation detector,
so that said frontal radiation detector gives at least, after said single vertical scanning:
   a first frontal image corresponding to a first portion of energy which is received by said frontal radiation detector and which is below a first given energy threshold, called low energy frontal image,
   a second frontal image corresponding to a second portion of energy which is received by said frontal radiation detector and which is above a second given energy threshold, called high energy frontal image,
   at least a combined frontal image corresponding to a combination of said first frontal image and said second frontal image,
and so that a lateral multi-energy image is made by performing a vertical scanning of a standing patient along said vertical scanning direction by said lateral radiation source and by said lateral radiation detector,
   with a driving current intensity modulation of said lateral radiation source, with no voltage intensity modulation of said lateral radiation source, depending on said patient bone thickness, on said patient soft tissue thickness, and depending on said identified specific bone(s) localization at different positions along said vertical scanning direction, which is performed automatically, so as to improve a compromise between:
      the global radiation dose received by a patient during said vertical scanning,
      and the local image contrasts of said identified specific bone(s) localization at different imaging positions along said vertical scanning direction, for, at least one or more, or preferably all, lateral images given by said lateral radiation detector,
so that said lateral radiation detector gives at least:
   a first lateral image corresponding to a first portion of energy which is received by said lateral radiation detector and which is below a first given energy threshold, called low energy lateral image,
   a second lateral image corresponding to a second portion of energy which is received by said lateral radiation detector and which is above a second given energy threshold, called high energy lateral image,
   at least a combined lateral image corresponding to a combination of said first lateral image and said second lateral image,
both frontal and lateral multi-energy images being made during same vertical scanning.

The invention not only deals with the formerly listed radiological methods, but also with radiological apparatuses implementing respectively these radiological methods.

In all preceding objects of the invention, except if mention to the contrary, a combined image (whether frontal, lateral, or both) corresponding to a combination of said first image and said second image, only means that this combined image is equal to the result of a combination of said first image and said second image, not that it has been obtained that way. The detector can, for instance, directly give the combined image and the second image, and then the first image is obtained by subtracting the second image from the combined image.

Preferred embodiments comprise one or more of the following features, which can be taken separately or together, either in partial combination or in full combination, with any of the preceding objects of the invention.

Preferably,
said first and second frontal scout views are processed to a multi-material decomposition with at least two material thickness vertical profiles,
   preferably, either a bi-material decomposition between Al and PMMA, or a bi-material decomposition between HA (HydroxyApatite) and H₂O,
and/or said first and second lateral scout views are processed to a multi-material decomposition with at least two material thickness vertical vectors,
   preferably, either a bi-material decomposition between Al and PMMA, or a bi-material decomposition between HA and H₂O,

Indeed, on one side Al or HA present X-ray attenuation properties close to human bone, whereas on the other side, PMMA or H₂O present X-ray attenuation properties close to human soft tissue.

Preferably,
said frontal mono-energy scout view is made by performing a single preliminary vertical scanning of a standing patient along said vertical scanning direction by said frontal radiation source and by said frontal radiation detector,
said lateral mono-energy scout view is made by performing a single preliminary vertical scanning of a standing patient along said vertical scanning direction by said lateral radiation source and by said lateral radiation detector,
both said frontal mono-energy scout view and said lateral mono-energy scout view being made during same single vertical scanning.

Hence, since frontal and lateral scout views are both performed simultaneously during same single vertical scanning, frontal and lateral scout views will topologically correspond exactly to each other, because the standing patient would not have moved in between.

Preferably, said first given energy threshold is equal or less than said second given energy threshold, preferably equal to said second given energy threshold.

Hence, in both cases all the range of energy threshold is covered, and in the second case at lower cost.

Preferably,
said first given energy threshold is equal to said second given energy threshold,
said frontal and/or lateral multi-energy images are made so that said frontal and/or lateral radiation detectors first give:
   said first frontal image,
   a third frontal image corresponding to the whole energy which is received by said frontal radiation detector, called total energy frontal image,
      said second frontal image being obtained by a subtracting said first frontal image from said third frontal image,
   and/or said first lateral image,
   and/or a third lateral image corresponding to the whole energy which is received by said lateral radiation detector, called total energy lateral image,
      said second lateral image being obtained by a subtracting said first lateral image from said third lateral image.

Hence, total energy image and high energy image can be given directly by the detector, whereas low energy image can be obtained by a simple subtraction, by subtracting high energy image from total energy image.

Preferably,
said frontal image is made by performing a vertical scanning of a standing patient along said vertical scanning direction by said frontal radiation source and by said frontal radiation detector, with:
   said modulation of a driving current intensity of at least said frontal radiation source along said vertical scanning direction, depending on said patient thickness and on said specific bone(s) localization at different positions along said vertical scanning direction,
said lateral image is made by performing a vertical scanning of a standing patient along said vertical scanning direction by said lateral radiation source and by said lateral radiation detector, with:
   said modulation of a driving current intensity of at least said lateral radiation source along said vertical scanning direction, depending on said patient thickness and on said specific bone(s) localization at different positions along said vertical scanning direction,
both said frontal image and said lateral image being made during same vertical scanning.

Hence, since frontal and lateral images are both performed simultaneously during same vertical scanning, frontal and lateral images will topologically correspond exactly to each other, because the standing patient would not have moved in between.

Preferably,
said driving current intensity modulation(s) of said frontal and/or lateral radiation source(s), with no voltage intensity modulation of said frontal and/or lateral radiation source(s), is performed automatically, so as to improve a compromise between:
   lowering the global radiation dose received by a patient during said vertical scanning,
   and not degrading under a given contrast threshold the local image contrasts of said identified specific bone(s) localization at different imaging positions along said vertical scanning direction, for all or part of patient thicknesses along said vertical scanning direction, for the frontal and/or lateral image(s).

This means that it is thereby possible:
either to go toward the lowest possible global radiation dose while keeping correct predetermined image quality,
or to improve image quality, while not raising too much the global radiation dose,
or even, that the margin given by the invention can be distributed, partly in order to lower the radiation dose, partly in order to keep a predetermined image quality.

Preferably,
said driving current intensity modulation(s) of said frontal and/or lateral radiation source(s), with no voltage intensity modulation of said frontal and/or lateral radiation source(s), is performed automatically, so as to improve a compromise between:
   lowering the global radiation dose received by a patient during said vertical scanning,
   and improving the contrast to noise ratio or the ratio between contrast to noise ratio and square root of said global radiation dose of said identified specific bone(s) localization at different imaging positions along said vertical scanning direction, with respect to local image contrasts of said identified specific bone(s) localization at different imaging positions along said vertical scanning direction with same global radiation dose but without any driving current intensity modulation, for all or part of patient thicknesses along said vertical scanning direction, for the frontal and/or lateral image(s).

This means that it is thereby possible:
either to go toward the lowest possible global radiation dose while keeping a predetermined good image quality,
or to improve deeply image quality, while not raising the global radiation dose,
or even, that the margin given by the invention can be distributed, partly in order to lower the radiation dose, partly in order to keep a better predetermined image quality.

Preferably,
said frontal multi-energy image acquisition is performed with at least 2 energy bins, or with at least 3 energy bins, or with at least 6 energy bins,
   and/or at most 20 energy bins, or at most 15 energy bins, or at most 10 energy bins,
and/or said lateral multi-energy image acquisition is performed with at least 2 energy bins, or with at least 3 energy bins, or with 6 energy bins,
   and/or at most 20 energy bins, or at most 15 energy bins, or at most 10 energy bins.

Hence, the higher the number of bins, the more accurately different tissue textures within the patient body can be distinguished from one another, but at the cost of an increasing complexity of the system, and with the risk that less useful signal becomes available for each bin.

Preferably,
for each said radiation detector:
   a radiation detector pixel size ranges from 50 μm to 250 μm, or ranges from 80 μm to 150 μm, or is about 100 μm,
   and/or the total height of radiation detector ranges from 0.1 cm to 1.2 cm, or from 0.2 cm to 1.0 cm, or from 0.3 cm to 0.7 cm,
   and/or the total width of radiation detector ranges from 10 cm to 80 cm, or from 20 cm to 70 cm, or from 30 cm to 60 cm,
and/or, said radiation detector can work in a Time Delay Summation mode.

Hence, the resolution of the image will be better, without creating too many artefacts, and the total useful width of the patient can be encompassed.

Preferably, said identified specific bone(s) localization includes a patient spine, preferably is a patient spine.

Indeed, patient spine is the specific bone(s) localization which is the most interesting to analyze in detail within a patient body, for orthopedic imaging purposes; therefore it is used to drive current intensity modulation.

Alternatively, the specific bone(s) localization may also be a pelvis or an arm or a leg of a standing patient along a vertical scanning direction, depending on the region of interest within the part of patient body which is imaged.

Preferably, said driving current intensity modulation of said frontal and/or lateral radiation source(s) is performed also so as to reach a value of signal to noise ratio which is constant and common to most of said imaging positions along said vertical scanning direction, preferably to all said imaging positions along said vertical scanning direction, for said frontal image and/or for said lateral image, but which can take two different values respectively for frontal image and for lateral image.

Preferably, for each of said frontal and/or lateral images, said signal to noise ratio value is constant and predetermined for each different patient organ to be imaged.

Preferably,
for a frontal image of a patient spine, said standard signal to noise ratio value corresponds to a number of X-ray photons received per detector pixel comprised between 50 and 70, the radiological imaging method operator preferably having the possibility to deviate, via a manual command, from this standard value by at least −25% or +100%, more preferably by at least −50% or +200%,
and/or for a lateral image of a patient spine, said standard signal to noise ratio value corresponds to a number of X-ray photons received per detector pixel comprised between 20 and 40, the radiological imaging method operator preferably having the possibility to deviate, via a manual command, from this standard value by at least −25% or +100%, more preferably by at least −50% or +200%.

Hence, with a constant and optimized signal to noise ratio along, or even all along, said vertical scanning direction, the local image contrasts of the identified specific bone(s) localization at different imaging positions along said vertical scanning direction are much improved, for what was indeed the region of interest within the frontal and/or lateral images.

Preferably, said frontal and/or lateral image, after having undergone at least said local image contrast improvements, is normalized by homogenization of raw radiations, in order to get rid of image artefacts coming from said driving current intensity modulation, and preferably wherein said frontal and/or lateral image, after having been normalized, undergoes a contrast enhancement step.

Indeed, because of this driving modulation, there were some artefacts in the frontal and/or lateral images, which were superimposing some modulation patterns of clear and dark grey levels on the image, rendering those images a bit less comfortable to interpret for the radiological imaging method operator.

Preferably, said identified specific bone(s) localization excludes metallic parts, if any, as for example metallic prosthesis of part of skeleton of patient body or as for example metallic protections put in place on patient body before performing said radiological imaging method.

Indeed, these foreign (to patient body) objects introduced within or on patient body, since being metallic and therefore stopping much more radiation (X-ray), than the rest of patient body, can lead to some bad optimization of the emitted dose, risking to lead, for the altitudes corresponding to these foreign objects, to some over exposure to emitted radiation. Where driving voltage intensity is constant, if metal outliers are not excluded, consequences can be worse since more or all parameters are chosen for a maximal thickness, leading to emitting a radiation dose higher or much higher than needed, that would be very detrimental to patient.

Preferably, said current intensity modulation is maximized so as to also maximize said vertical scanning speed at a constant value.

Hence, for a given emitted radiation dose, so for a given radiation dose received by standing patient during said vertical scanning, both kept at same level, the total vertical scanning time is notably reduced, having the advantage of lowering the possibility for the standing patient to move and the effects of a patient motion, thereby reducing somewhat the risk of blurring and the risk of deformation of the frontal and lateral images.

Preferably, said operating mode can be either switched on or switched off manually by a radiological imaging method operator.

Hence, this very advantageous way of operating a radiological imaging apparatus is available, whereas it can be cancelled if and when the operator of this radiological imaging apparatus wants to get rid of it, in order for instance to fully manually operate this radiological imaging apparatus. The radiological imaging method according to advantageous embodiments of the invention presents 3 operating modes: a full manual mode, an AEC mode (AEC=Automatic Exposure Control) without modulation, an AEC mode with modulation.

Preferably, said operating mode can be used for patient morphotypes ranging from children to obese adults, and is dedicated to vertical scanning of large and/or obese patients, and/or wherein said operating mode is dedicated to vertical scanning of children patients.

The radiological imaging method according to the invention is all the more interesting when the thickness of the patient can be especially lower or especially higher than for an averaged size patient. This shows the capability of the radiological imaging method according to the invention to be very patient specific and account for wide attenuation ranges in long axis imaging. Of course, the radiological imaging method according to the invention works also very well for standard sized patients.

Preferably, said current intensity modulation(s) rate do(es) not go beyond a predetermined threshold of 5 mA per millisecond, or do(es) not go beyond a predetermined threshold of 2 mA per millisecond, or do(es) not go beyond a predetermined threshold of 1 mA per millisecond.

Hence, the radiological imaging method according to the invention can be performed also with relatively simple and cheap radiation sources with relatively slow current intensity driving capabilities.

Preferably, said current intensity modulation(s) at least range(s) from 20 mA to 300 mA, and preferably from 10 mA to 400 mA.

Hence, the radiological imaging method according to the invention can be performed also with relatively simple and cheap radiation sources with relatively limited ranges of current intensity driving capabilities.

Preferably, said vertical scanning speed value at least range(s) from 8 cm/second to 20 cm/second, and preferably from 0.4 cm/second to 35 cm/second.

Hence, the radiological imaging method according to the invention can be performed also with relatively simple and cheap radiation sources with relatively limited extent of ranges of vertical scanning speed capabilities, while at the same time fully taking advantage of available ranges of vertical scanning speed capabilities.

Preferably, each of said frontal and/or lateral scout view(s) is made by performing a preliminary vertical scanning of a standing patient along a vertical scanning direction with a reduced global radiation dose as compared to each of said frontal and lateral images, before making each of said frontal and lateral images, and preferably wherein said reduced global radiation is less than 10% of said global radiation dose, more preferably less than 5% of said global radiation dose.

Hence, depending on the thickness profile(s) and on the specific bone(s) localization of patient standing body along the vertical scanning direction, the modulation of driving current intensity, as well as possibly of vertical scanning speed, can be determined just before performing the vertical scanning which will result in effective frontal and lateral images of standing patient body performed with a limited but full radiation dose sufficient to make high quality frontal and lateral images. The scout view(s) can be performed at the cost of quite a limited over exposure to emitted radiation. The benefit can even be double: not only is the over exposure during scout view performance (+10% or +5%) very limited, but also it is very efficient to optimize compromise between global radiation dose received and enhancement of image contrast.

Preferably, pixels in said scout view are gathered together, preferably by zones of N×N pixels, more preferably by zones ranging from 2×2 pixels to 10×10 pixels, to make imaged zones.

Hence, image quality and image contrast are enhanced for the scout view, despite the very low level of emitted radiation dose used to perform this scout view.

Preferably, said images or said imaged zones are processed to identify salient points which in turn are used to compute said thickness profile and to identify said specific bone(s) localization of a standing patient along said vertical scanning direction.

Hence, it is easier and more efficient to compute said thickness profile(s) and to identify said specific bone(s) localization of a standing patient along the vertical scanning direction, from the scout view, despite the very low level of emitted radiation dose.

Preferably, said images or said imaged zones are processed by a neural network to compute said thickness profile and to identify said specific bone(s) localization of a standing patient along said vertical scanning direction.

Hence, it is easier and more efficient to identify said specific bone(s) localization of a standing patient along the vertical scanning direction, from the scout view, despite the very low level of emitted radiation dose.

Preferably, said 2 radiation sources slide vertically so as to perform vertical scanning of a pelvis or of a spine or of a full body of a standing patient along a vertical scanning direction.

Preferably, said 2 radiation detectors are respectively associated with said 2 radiations sources, said 2 radiation detectors being 2 Photon Counting Detectors (PCD) each being associated to an automatic image processing function automatically balancing image density whatever radiation dose received on the sensitive surface of said radiation detector to homogenize responses of said detectors.

This is an interesting function since it would be harder for the radiological imaging method operator to correctly assess manually over exposure or under exposure to radiation signal emitted by radiation sources. Besides, Photon Counting Detectors present improved linearity and signal to noise ratio, as compared to gaseous detectors.

Preferably, said 2 radiation detectors are respectively associated with said 2 radiations sources, said 2 radiation detectors being 2 multi-energy counting detectors, preferably being 2 Energy Resolved Photon Counting Detectors (ERPCD).

Preferably, radiation is X-ray.

A standing patient or a patient in a standing position is a patient who is in a weight bearing position, contrary to a lying patient or to a patient who is in a lying position as in computed tomography. Another patient weight bearing position, alternative to patient standing position could be a patient seating position.

Preferably, the voltage intensity of said frontal radiation source is more than 90 kVp, or more preferably more than 100 kVp.

Hence, the distinction between bone and soft tissue within the patient, will become easier.

Preferably, said second energy threshold is chosen so as to improve image contrast more for lower patient thicknesses regions along vertical direction than for higher patient thicknesses regions along vertical direction, preferably said second energy threshold being chosen between 50 keV and 90 keV, preferably between 60 keV and 80 keV, more preferably said second energy threshold being chosen at 70 keV.

Hence, even if wide patient chests can still be seen accurately, narrow arms or legs will not at all or practically not be sacrificed by being not saturated or very slightly saturated, i.e. over exposed due to system limitations.

Preferably, said first energy threshold and/or said second energy threshold are modified, and/or an associated spectral filtration, preferably k-edge filtration, is used and tuned, depending on said patient thickness and/or on said patient specific bone localization at different imaging positions along said vertical scanning direction.

Hence, accuracy of end-user image used by practitioner is even improved.

Preferably,
said second frontal image includes information which allows for assessing a patient bone density, and said second lateral image includes information which allows for assessing a patient bone density,
and/or said combined frontal image presents local image contrasts of said identified specific bone(s) localization at different imaging positions along said vertical scanning direction which are sufficient to perform a diagnostic on a patient, and said combined lateral image presents local image contrasts of said identified specific bone(s) localization at different imaging positions along said vertical scanning direction which are sufficient to perform a diagnostic on a patient.

Hence, with one global operation, two different kinds of images have been performed, leading to two different useful applications by the practitioner.

To all preceding objects of the invention and/or to all preceding combinations, can be added either a mechanical cross scattering correction with vertical gap, as described for example in patent applications EP 16711889 or U.S. Pat. No. 1,607,660, and/or a software cross scattering correction, as described for example in patent applications EP 17758269 or U.S. Pat. No. 16,628,410.

Preferably the multi-energy image is a dual-energy image.

Further features and advantages of the invention will appear from the following description of embodiments of the invention, given as non-limiting examples, with reference to the accompanying drawings listed hereunder.

### BRIEF DESCRIPTION OF THE DRAWINGS

FIG. 1 shows an example of an imaging workflow of the radiological imaging method according to an embodiment of the invention, with current modulation but without voltage modulation.

FIG. 2 shows an example of multi-energy scout view acquisition step within the radiological imaging method according to an embodiment of the invention and/or of multi-energy scan image acquisition step within the radiological imaging method according to an embodiment of the invention.

FIG. 3 shows another example of multi-energy scout view acquisition step within the radiological imaging method according to an embodiment of the invention and/or of multi-energy scan image acquisition step within the radiological imaging method according to an embodiment of the invention.

FIG. 4 shows an example of bone localization computation step with profile of interest extraction within the radiological imaging method according to an embodiment of the invention.

FIG. 5 shows an example of a part of patient bone and soft tissue thickness computation step and/or of patient total thickness computation within the radiological imaging method according to an embodiment of the invention.

FIG. 6 shows a first example of another part of patient total thickness computation step within the radiological imaging method according to an embodiment of the invention.

FIG. 7 shows a second example of another part of patient bone and soft tissue thickness computation step within the radiological imaging method according to an embodiment of the invention.

FIG. 8 shows an example of a frontal patient mono-energy diagnostic image not according to an embodiment of the invention, without additional possibility of getting at a computation of a good quality bone density distribution image.

FIG. 9 shows an example of a frontal patient multi-energy (dual-energy) image according to an embodiment of the invention, with additional possibility of getting at a computation of a good quality bone density distribution image.

FIG. 10 shows a zoom of thorax region of FIG. 8.

FIG. 11 shows a zoom of thorax region of FIG. 9.

FIG. 12 shows an example of performance of bone density computation step within the radiological imaging method according to an embodiment of the invention.

FIG. 13 shows an example of successive images transformations during application of bone density computation step within the radiological imaging method according to an embodiment of the invention as on FIG. 12.

FIG. 14 shows an example of a table comparing bone density values between on the one side DXA images not according to an embodiment of the invention, without additional possibility of getting a good quality diagnostic, versus bone density images according to an embodiment of the invention, with additional possibility of getting at a computation of a good quality diagnostic.

FIG. 15 shows an example of structure of imaging device to implement the radiological imaging method according to an embodiment of the invention.

### DETAILED DESCRIPTION OF THE INVENTION

In the following description, without mention to the contrary, what is said from frontal scout view or image can be applied similarly respectively to lateral scout view or image, and vice-versa. All what is done both for frontal and lateral scout view can be done either for frontal scout view only or for lateral scout view only, if only frontal scout view or only lateral scout view is of interest to practitioner or to patient. All what is done both for frontal and lateral image can be done either for frontal image only or for lateral image only, if only frontal image or only lateral image is of interest to practitioner or patient. Wherever a profile is mentioned, except mention to the contrary, a sequence of several partial or regional mean values (a mean value per zone or organ, for instance, leg, pelvis, spine, neck, head, or portions of those) or sometimes a single mean value may be sufficient, although results will or may be less accurate.

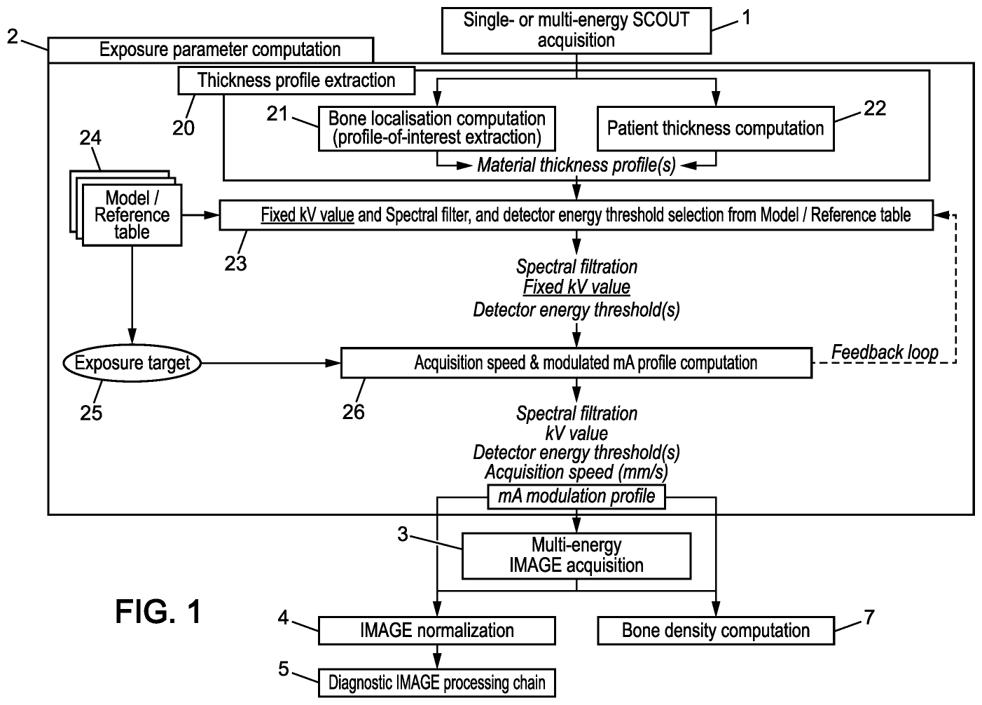

FIG. 1 shows an example of an imaging workflow of the radiological imaging method according to an embodiment of the invention, with current modulation but without voltage modulation.

In a first alternative, a multi-energy image, and even a dual-energy image is performed based on a mono-energy scout view acquisition.

First, a step 1 of acquisition of a frontal scout view and a lateral scout view is performed.

Then, a step 20 of thickness profile extraction is performed from the frontal scout view and the lateral scout view performed at acquisition step 1. This step 20 includes a sub-step 21 of bone localization computation with a profile of interest extraction along patient height performed in parallel and simultaneously to a sub-step 22 of computation of a patient thickness, then a patient thickness profile along patient height is processed by extraction of thickness values along the profile of interest coordinates. At the end of thickness profile extraction step 20, are available for next step which is a determination and selection step 23, a patient thickness profile along patient height. The patient thickness profile along patient height corresponds to the total thickness, including both patient bone thickness and patient soft-tissue thickness, along patient height.

Then, a step 23 of determination of fixed voltage value (in kV) and of spectral filtration, and also of selection of detector energy threshold(s), from a model and/or reference table 24 which includes a catalog of models and/or of references, based on the patient thickness profile along patient height. The model and/or reference table 24 gives an exposure target 25 which corresponds to the model or to the reference which has been selected in determination and selection step 23. At the end of determination and selection step 23, there are available for next computation step 26, a spectral filtration, a fixed voltage value, one or more detector energy threshold(s).

Then, a computation step 26 performs computation of an acquisition speed for the vertical scanning along the height of the standing (or seating) patient and of a current modulation profile along patient height. Computation step 26 also uses the exposure target 25. Computation step also performs a feedback loop toward determination and selection step 23. At the end of computation step 26, are available for next image acquisition step 3, a spectral filtration, a fixed voltage value, one or more detector energy threshold(s), a vertical scanning acquisition speed (in mm per second), a current modulation profile in mA along patient height. The current modulation profile along patient height, as well as the other parameters, will also be directly available for the image normalization step 4 and for the bone density computation step 7.

In this computation step 26 performs computation of an acquisition speed for the vertical scanning along the height of the standing (or seating) patient and of a current modulation profile along patient height, the current profile and the vertical scan speed selection strategy, for a given voltage value, is similar to the strategy used in patent application WO2021/094806 (hereby incorporated by reference), in summary:
computation of an exposure budget (the needed product of the current value by the exposure time to reach the exposure target),
maximization of the vertical scan speed, given the available tube current budget (constrained by hardware specifications in terms of max tube power and max instantaneous current value),
computation of the corresponding current profile therefrom.

Given an optimal voltage value for a set of parameters \vec{p} (including thickness profiles), the product of the current value by the exposure time (in mAs) that is required in order to reach target signal S_{T} is

$$mA s = \frac{{S}_{T}}{\overline{S} ( kV , \vec{p} )}$$

The exposure time t is given by the highest current value that can be used for the given voltage value, when accounting for tube power limits and lower as well as upper current bounds. The acquisition speed is selected (as a free value or among a collection of admissible system speeds) as the smallest speed among the computed speeds along the thickness profiles. The current values are then updated to match the target signal, given this fixed acquisition speed.

Wherever the current values fall below, respectively beyond the current bounds, voltage may be decreased, respectively increased, in order to bring them back to the admissible range of values, until voltage values themselves reach their lower and/or upper bounds. Matching the target signal is preferred over keeping the optimal voltage value, this preference being met thanks to the feedback loop from this step 26 to step 23 (see FIG. 1).

Once the optimal exposure parameters are selected, their profiles along the slot scan may further be adapted to hardware constraints (e.g., on the slopes of the current and voltage profiles) if these constraints were not yet accounted for in the former steps. The exposure parameters are then be formatted in files that are readable by the system and an image acquisition step 3 can be performed (see FIG. 1).

All steps 20, 23, 24, 25, 26 are part of an exposure parameter computation routine 2.

Then, after this exposure parameter computation routine 2 has been fully completed, an image acquisition step 3 is performed, based on spectral filtration, fixed voltage value, one or more detector energy threshold(s), vertical scanning acquisition speed (in mm per second), current modulation profile (in mA) along patient height. This acquired image is a multi-energy image, and even here a dual-energy image. The acquired image can include a frontal image and/or a lateral image. The acquired image includes preferably a frontal image and a lateral image.

Based upon the acquired multi-energy images at acquisition step 3 and on the current intensity modulation profile, the frontal and lateral acquired images are then normalized in a normalization step 4. The frontal and lateral normalized images can then be further processed in a way, (with post-processing steps) and/or afterwards displayed on a screen to be seen by the practitioner, in a diagnostic image processing chain step 5, so as to give to practitioner a diagnostic good quality image of patient.

The diagnostic image processing chain 5 corresponds to the standard image processing chain used with single-energy image acquisitions usually, but applied to a combination of low-energy and high-energy normalized images, i.e. to the sum of low-energy and high-energy images.

Based upon the acquired multi-energy images at acquisition step 3 and on the current intensity modulation profile, and on the other available parameters, the frontal and lateral acquired images are then further processed in another way (with other post-processing steps), in a bone density computation step 7, so as to give to practitioner a bone density computation distribution which can also be displayed for practitioner.

In a second alternative, a multi-energy image, and here even a dual-energy image is performed based on a multi-energy, and even a dual-energy, scout view acquisition.

First, a step 1 of acquisition of a frontal scout view and a lateral scout view is performed.

Then, a step 20 of thickness profile extraction is performed from the frontal scout view and a lateral scout view performed at acquisition step 1. This step 20 includes a sub-step 21 of bone localization computation with a profile of interest extraction along patient height performed in parallel and simultaneously to a sub-step 22 of computation of both a patient bone thickness and of a patient soft tissue thickness, then both a patient bone thickness profile and of a patient soft tissue thickness profile along patient height are processed by extraction of respective thicknesses values along the profile of interest coordinates. In sub-step 21, coordinates {x_i} (x_1, x_2 . . . x_N), corresponding to localization of points in the profile of interest, are extracted from the scout views. In sub-step 22, conversions between signals and thicknesses are performed, which result in a bone thickness image fBone and in a soft tissue thickness image fSoft. Combined result of sub-steps 21 and 22 is the collection of {fBone(x_i)} and of {fSoft(x_i)}.

At the end of thickness profile extraction step 20, both a patient bone thickness profile and of a patient soft tissue thickness profile along patient height are available for next step which is a determination and selection step 23.

Then, a step 23 of determination of fixed voltage value (in kV) and of spectral filtration, and also of selection of detector energy threshold(s), from a model and/or reference table 24 which includes a catalog of models and/or of references, based on the patient bone thickness profile and on a patient soft tissue thickness profile along patient height. The model and/or reference table 24 gives an exposure target 25 which corresponds to the model or to the reference which has been selected in determination and selection step 23. At the end of determination and selection step 23, a spectral filtration, a fixed voltage value, one or more detector energy threshold(s) are available for next computation step 26.

Then, a computation step 26 computes an acquisition speed for the vertical scanning along the height of the standing (or seating) patient and of a current modulation profile along patient height. Computation step 26 also uses the exposure target 25. Computation step also performs a feedback loop toward determination and selection step 23. At the end of computation step 26, a spectral filtration, a fixed voltage value, one or more detector energy threshold(s), a vertical scanning acquisition speed (in mm per second), a current modulation profile in mA along patient height, are available for next image acquisition step 3. The current modulation profile along patient height will also be directly available for the image normalization step 4 and for the bone density computation step 7.

In this computation step 26 performs computation of an acquisition speed for the vertical scanning along the height of the standing (or seating) patient and of a current modulation profile along patient height, the current profile and the vertical scan speed selection strategy, for a given voltage value, is similar to the strategy used in patent application WO2021/094806 (hereby incorporated by reference), in summary:
computation of an exposure budget (the needed current value multiplied by the exposure time to reach the exposure target),
maximization of the vertical scan speed, given the available tube current budget (constrained by hardware specifications in terms of min and max tube powers and min and max instantaneous current values),
computation of the corresponding current profile therefrom.

Given an optimal voltage value for a set of parameters \vec{p} (including thickness profiles), the product of current value by exposure time (in mAs: milliamperes seconds) that is required in order to reach target signal S_{T} is

$$mA s = \frac{{S}_{T}}{\overline{S} ( kV , \vec{p} )}$$

The exposure time t is given by the highest current value that can be used for the given voltage value, when accounting for tube power limits and lower as well as upper current bounds. The acquisition speed is selected (as a free value or among a collection of admissible system speeds) as the smallest speed among the computed speeds along the thickness profiles. The current values are then updated to match the target signal, given this fixed acquisition speed.

Wherever the current values fall below, respectively beyond the current bounds, voltage may be decreased, respectively increased, in order to bring them back to the admissible range of values, until voltage values themselves reach their lower and/or upper bounds. Matching the target signal is preferred over keeping the optimal voltage value, this preference being met thanks to the feedback loop from this step 26 to step 23 (see FIG. 1).

Once the optimal exposure parameters are selected, their profiles along the slot scan may further be adapted to hardware constraints (e.g., on the slopes of the current and voltage profiles) if these constraints were not yet accounted for in the former steps. The exposure parameters are then be formatted in files that are readable by the system and an image acquisition step 3 can be performed (see FIG. 1).

All steps 20, 23, 24, 25, 26 are part of an exposure parameter computation routine 2.

Then, after this exposure parameter computation routine 2 has been fully completed, an image acquisition step 3 is performed, based on spectral filtration, fixed voltage value, one or more detector energy threshold(s), vertical scanning acquisition speed (in mm per second), current modulation profile (in mA) along patient height. This acquired image is a multi-energy image, and even a dual-energy image. The acquired image can include a frontal image and/or a lateral image. The acquired image includes preferably a frontal image and a lateral image.

Based upon the acquired multi-energy images at acquisition step 3 and on the current intensity modulation profile, as well as on the other available parameters, the frontal and lateral acquired images are then normalized in a normalization step 4. The frontal and lateral normalized images can then be further processed in a way, (with post-processing steps) and/or afterwards displayed on a screen to be seen by the practitioner, in a diagnostic image processing chain step 5, so as to give to practitioner a diagnostic quality image of patient.

The diagnostic image processing chain 5 corresponds to the standard image processing chain used with single-energy image acquisitions usually, but applied to a combination of low-energy and high-energy normalized images, i.e. to the sum of low-energy and high-energy images.

Based upon the acquired multi-energy images at acquisition step 3 and on the current intensity modulation profile, as well as on the other available parameters, the frontal and lateral acquired images are then further processed in another way (with other post-processing steps), in a bone density computation step 7, so as to give to practitioner a bone density computation distribution which can also be displayed for practitioner.

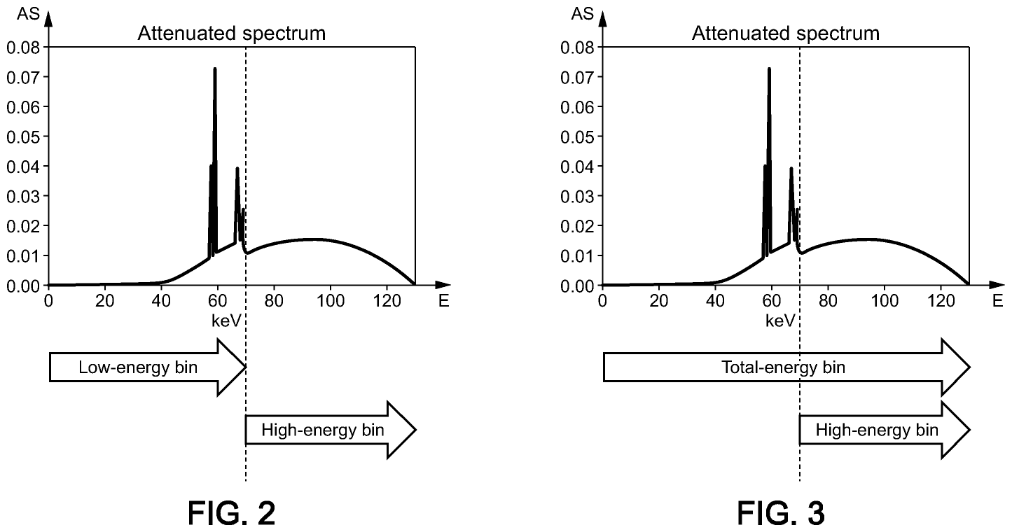

FIG. 2 shows an example of multi-energy scout view acquisition step within the radiological imaging method according to an embodiment of the invention and/or of multi-energy scan image acquisition step within the radiological imaging method according to an embodiment of the invention.

In multi-energy scout view acquisition step 1 in FIG. 1, a normalized attenuated spectrum AS is represented as a function of an energy E expressed in keV.

In multi-energy scout view mode, the detector reads low-energy and high-energy scout views directly, and deduces the total-energy scout view by adding together the high-energy scout view and the low-energy scout view.

In mono-energy or single-energy scout view mode, there would be only a single bin which would correspond to the total energy bin.

In multi-energy image acquisition step 3 in FIG. 1, a normalized attenuated spectrum AS is represented as a function of an energy E expressed in keV.

In multi-energy image acquisition, the detector reads low-energy and high-energy images directly, and deduces the total-energy image by adding together the high-energy image and the low-energy image.

FIG. 3 shows another example of multi-energy scout view acquisition step within the radiological imaging method according to an embodiment of the invention and/or of multi-energy scan image acquisition step within the radiological imaging method according to an embodiment of the invention.

In multi-energy scout view acquisition step 1 in FIG. 1, an attenuated spectrum AS is represented as a function of an energy E expressed in keV.

In multi-energy scout view mode, the detector reads a total-energy scout view and a high-energy scout view, and deduces the low-energy scout view by subtracting the high-energy scout view from the total-energy scout view.

In mono-energy or single-energy scout view mode, there would be only a single bin which would correspond to the total energy bin.

In multi-energy image acquisition step 3 in FIG. 1, an attenuated spectrum AS is represented as a function of an energy E expressed in keV.

In multi-energy image acquisition, the detector reads a total-energy image and a high-energy image, and deduces the low-energy image by subtracting the high-energy image from the total-energy image.

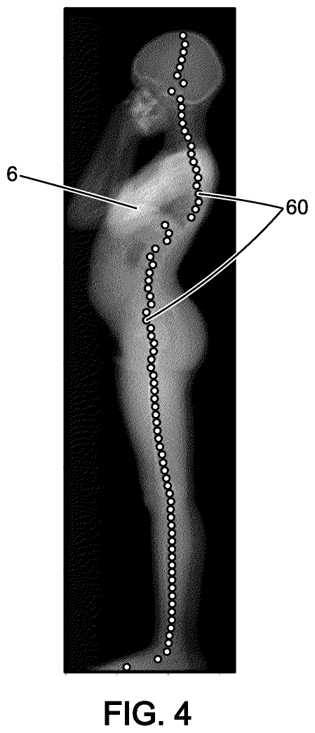

FIG. 4 shows an example of bone localization computation step with profile of interest extraction within the radiological imaging method according to an embodiment of the invention.

In FIG. 4, there is a scout view 6, here a lateral scout view 6, with salient points 60 located along the lateral image 6 of the patient skeleton, the salient points 60 representing the patient specific bone localization at different imaging positions along said vertical scanning direction which is also standing patient height here.

These salient points 60 represent a curve which has been extracted from the patient lateral image 6 by summarizing the detector signal values in anatomical landmarks with clinical interest along the slot scan. Therefore, several processing techniques can be used, which can be based on image filtering and segmentation techniques, or on landmark extraction from machine learning techniques.

This is a way to perform the sub-step 21 of bone localization computation.

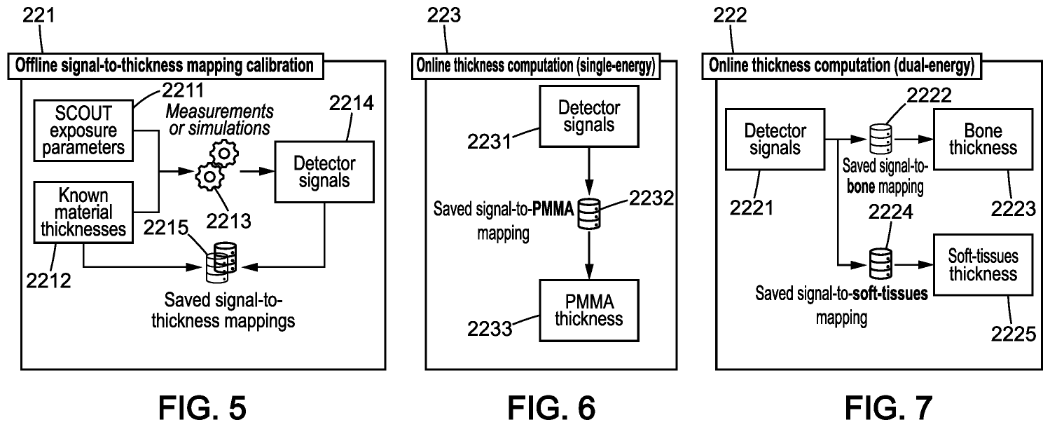

FIG. 5 shows an example of a part of patient bone and soft tissue thickness computation step and/or of patient total thickness computation within the radiological imaging method according to an embodiment of the invention.

In a first alternative, multi-energy case, the sub-step 22 of computation of both a patient bone thickness and of a soft tissue thickness is performed each time by an online operation 222 of online thickness computation 222 on FIG. 7 using information got separately offline by an offline operation 221 of offline signal-to-thickness mapping calibration on FIG. 5.

In the offline operation 221 of offline signal-to-thickness mapping calibration, scout exposure parameters 2211 and known material thicknesses 2212 are used as inputs to a measurement or simulation 2213 which gives as output measured or simulated detector signals 2214. Then, both known material thicknesses 2212 and measured or simulated detector signals 2214 are saved and stored in a signal-to-thickness mappings database 2215. The signal-to-thickness mappings database 2215, which is computed offline, includes mappings from detector signal counts to corresponding material thicknesses. These mappings can be for example look up tables (LUT) or model fits. Contrary to mono-energy scout view, where the mapping is based on the total-energy signal, the multi-energy scout view mapping, here the dual-energy scout view mapping is based on the signal pair, including both low-energy signal and high-energy signal, with computation of one mapping per material: soft tissue for low-energy signal, bone for high-energy signal. PMMA thicknesses are stored in a system of coordinates high energy and low energy. Al thicknesses are stored in a system of coordinates high energy and low energy.

In a second alternative, mono-energy case, the sub-step 22 of computation of a patient thickness is performed each time by an online operation 223 of online thickness computation 223 on FIG. 6 using information got separately offline by an offline operation 221 of offline signal-to-thickness mapping calibration on FIG. 5.

In the offline operation 221 of offline signal-to-thickness mapping calibration, scout exposure parameters 2211 and known material thicknesses 2212 are used as inputs to a measurement or simulation 2213 which gives as output measured or simulated detector signals 2214. Then, both known material thicknesses 2212 and measured or simulated detector signals 2214 are saved and stored in a signal-to-thickness mappings database 2215. The signal-to-thickness mappings database 2215, which is computed offline, includes mappings from detector signal counts to corresponding material thicknesses. These mappings can be for example look up tables (LUT) or model fits. Because of the mono-energy scout view, the mapping is based on the total-energy signal, corresponding to total signal, with computation of one mapping of total thickness for one material: soft tissue. PMMA thicknesses are stored in a system of coordinates high energy and low energy.

FIG. 6 shows a first example of another part of patient total thickness computation step within the radiological imaging method according to an embodiment of the invention, which is performed at the beginning of the mono-energy case.

In the online operation 223 of online thickness computation, measured detector signals 2231 are used, as input to a simplified database 2232 or to a part 2232 of database 2215, storing signal-to-PMMA thickness (corresponding to total thickness) mappings. The database 2232 storing signal-to-PMMA thickness mappings gives as output a patient total thickness 2233.

FIG. 7 shows a second example of another part of patient bone and soft tissue thickness computation step within the radiological imaging method according to an embodiment of the invention.

In the online operation 222 of online thickness computation, measured detector signals 2221 are used as input to both a sub-database 2222 storing signal-to-bone thickness mappings and to a sub-database 2224 storing signal-to-soft-tissue thickness mappings. The sub-database 2222 storing signal-to-bone thickness mappings gives as output a bone thickness 2223. The sub-database 2224 storing signal-to-soft-tissue thickness mappings gives as output a soft-tissue thickness 2225.

FIG. 8 shows an example of a frontal patient mono-energy diagnostic image not according to an embodiment of the invention, without additional possibility of getting at a computation of a good quality bone density distribution image. A frontal diagnostic image 80 of good quality with a thorax region 81 showing patient spine.

FIG. 9 shows an example of a frontal patient multi-energy (dual-energy) image according to an embodiment of the invention, with additional possibility of getting at a computation of a good quality bone density distribution image. A frontal diagnostic image 90 of good quality with a thorax region 91 showing patient spine.

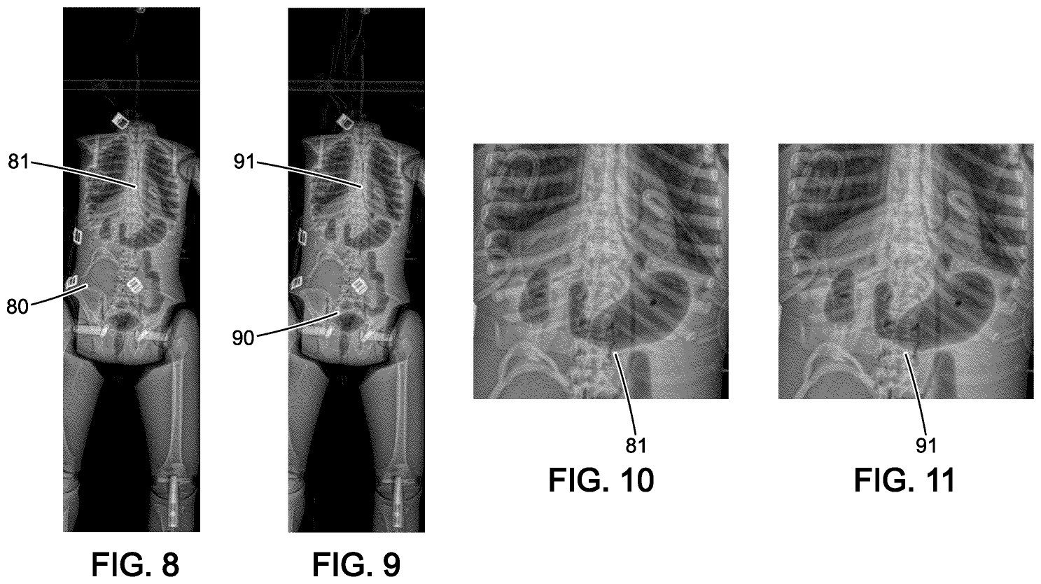

FIG. 10 shows a zoom of thorax region of FIG. 8. The thorax region 81 is represented by a diagnostic frontal image of good quality derived from an acquired frontal image, but no additional computation of a good quality bone density distribution can be derived from this acquired frontal image.

FIG. 11 shows a zoom of thorax region of FIG. 9. The thorax region 91 is represented by a diagnostic frontal image of good quality derived from an acquired frontal image, and besides, an additional computation of a good quality bone density distribution can be derived from this acquired frontal image.

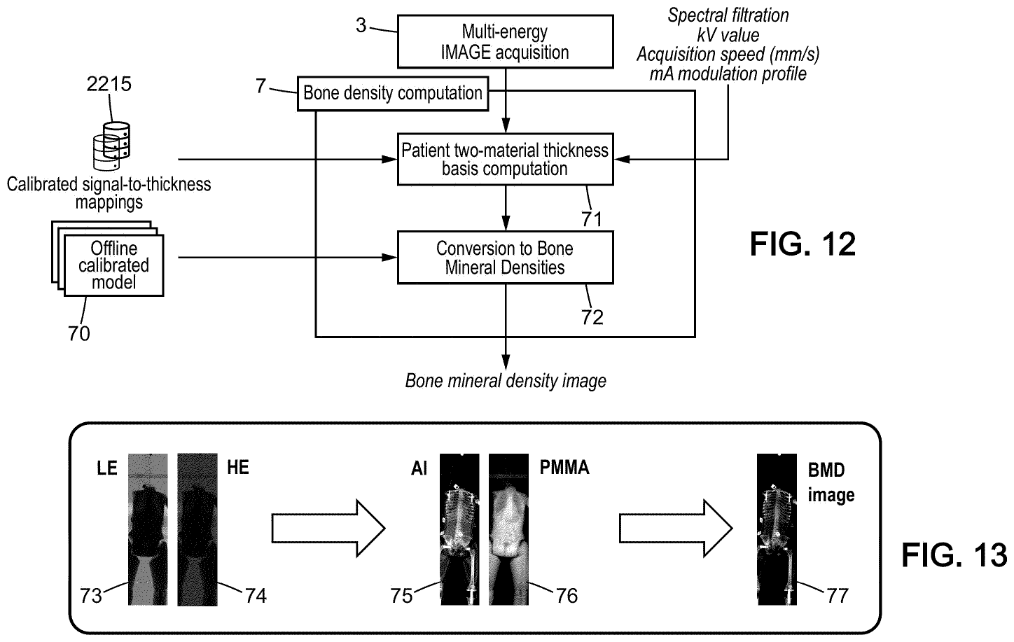

FIG. 12 shows an example of performance of bone density computation step within the radiological imaging method according to an embodiment of the invention.

FIG. 13 shows an example of successive images transformations during application of bone density computation step within the radiological imaging method according to an embodiment of the invention as on FIG. 12.

When referring to FIG. 1, a bone density computation 7 can be performed after the multi-energy image acquisition step 3.

Spectral filtration, fixed voltage value, one or more detector energy threshold(s), vertical scanning acquisition speed (in mm per second), current modulation profile (in mA) along patient height, as well as the calibrated signal-to-thickness(es) mappings of the database 2215 (on FIG. 5), are available to the patient two material thickness basis computation sub-step 71 to get at a first transformation of the images 73 and 74, low-energy and high-energy images, acquired after multi-energy image acquisition step 3, into the images 75 and 76, Al and PMMA images.

Then, by using an offline calibrated model 70, these images 75 and 76, Al and PMMA images are transformed and combined into a BMD image 77 (bone mineral density image also simpler called bone density image), by the sub-step 72 of conversion to Bone Mineral Densities.

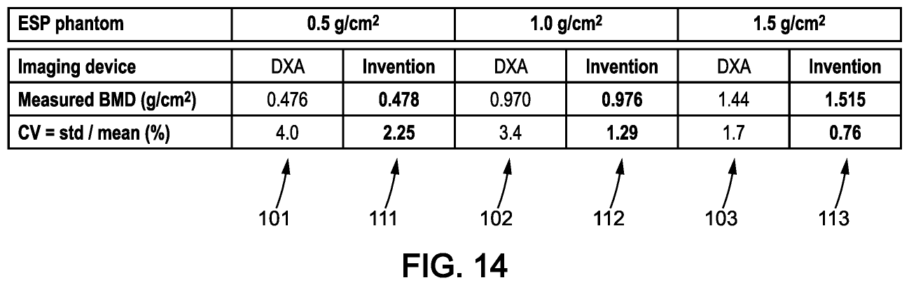

FIG. 14 shows an example of a table comparing bone density values between on the one side DXA images not according to an embodiment of the invention, without additional possibility of getting a good quality diagnostic, versus bone density images according to an embodiment of the invention, with additional possibility of getting at a computation of a good quality diagnostic.

The DXA columns 101, 102, 103, corresponding respectively for low, intermediate, high bone density, are extracted from a prior art paper “Nowak, T., Eberhard, M., Schmidt, B., Frey, D., Distler, O., Saltybaeva, N., . . . & Euler, A. (2021)” with “Bone mineral density quantification from localizer radiographs: accuracy and precision of energy-integrating detector CT and photon-counting detector CT. Radiology, 298 (1), 147-152”. The values are measured on the European Spine Phantom (ESP).

The bone mineral density images made by embodiments of the invention, are at least as good as, and even sometimes somewhat better than those of this prior art “Nowak et al (2021)”, as can be seen from the “invention columns” 111, 112, 113, corresponding respectively for low, intermediate, high bone density, by comparison with the DXA columns 101, 102, 103. Besides, with the multi-energy images acquired in step 3 according to an embodiment of the invention, a good quality diagnostic image can be additionally derived, what cannot be done with this prior art “Nowak et al (2021)”.

The multi-energy image acquisition according to embodiments of the invention allows for both:
derivation of a good quality image,
derivation of a good quality bone density image,
where processes according to prior art can only derive one of these two types of images, but not both such types of images from the same image acquisition step.

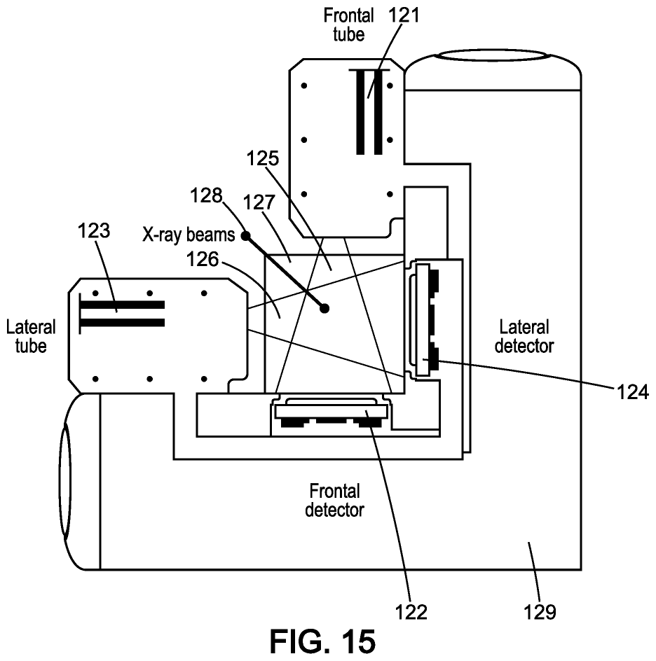

FIG. 15 shows an example of structure of imaging device to implement the radiological imaging method according to an embodiment of the invention. Vertical scanning direction is orthogonal to plan of FIG. 15. Patient height is also orthogonal to plan of FIG. 15. Vertical scanning direction is the patient height scanning direction, so a scanning direction along patient height with a standing patient.

There is an imaging apparatus or an imaging device 129.

A frontal emission and reception line includes a frontal tube 121 which emits an X-ray frontal beam 125 which goes through patient body (not represented here but located in intersection zone 128) and arrives on frontal detector 122.

A lateral emission and reception line includes a lateral tube 123 which emits an X-ray lateral beam 126 which goes through patient body (not represented here but located in intersection zone 128) and arrives on lateral detector 124.

To avoid cross scattering in intersection zone 128, mechanical correction with vertical gap between frontal and lateral emission and reception lines and/or software cross-scattering correction between frontal and lateral emission and reception lines can be used, as mentioned above.

The invention has been described with reference to preferred embodiments. However, many variations are possible within the scope of the invention.

1-39. (canceled)

40. A radiological imaging method comprising:
2 radiation sources with imaging directions orthogonal to each other, one frontal radiation and one lateral radiation source, sliding vertically so as to perform vertical scanning of a standing patient along a vertical scanning direction,
2 radiation detectors which are respectively associated with said 2 radiations sources, one frontal radiation detector and one lateral radiation detector, sliding vertically so as to perform vertical scanning of a standing patient along said vertical scanning direction, at least said frontal radiation detector being a multi-energy counting detector,
wherein said radiological method comprises at least one operating mode in which:
a frontal mono-energy scout view is made by performing a preliminary vertical scanning of a standing patient along said vertical scanning direction by said frontal radiation source and by said frontal radiation detector,
said frontal scout view is processed to identify a patient thickness and a specific bone(s) localization at different positions along said vertical scanning direction within said frontal scout view,
a driving current intensity of at least said frontal radiation source is modulated along said vertical scanning direction, depending on identified patient thickness and on said identified specific bone(s) localization at different positions along said vertical scanning direction,
so that a frontal multi-energy image is made by performing a single vertical scanning of a standing patient along said vertical scanning direction by said frontal radiation source and by said frontal radiation detector,
with a driving current intensity modulation of said frontal radiation source, with no voltage intensity modulation of said frontal radiation source, depending on said patient thickness and depending on said identified specific bone(s) localization at different positions along said vertical scanning direction, which is performed automatically, so as to improve a compromise between:
the global radiation dose received by a patient during said vertical scanning,
and the local image contrasts of said identified specific bone(s) localization at different imaging positions along said vertical scanning direction, for, at least one or more, or preferably all, frontal images given by said frontal radiation detector,
so that said frontal radiation detector gives at least, after said single vertical scanning:
a first frontal image corresponding to a first portion of energy which is received by said frontal radiation detector and which is below a first given energy threshold, called low energy frontal image,
a second frontal image corresponding to a second portion of energy which is received by said frontal radiation detector and which is above a second given energy threshold, called high energy frontal image,
at least a combined frontal image corresponding to a combination of said first frontal image and said second frontal image.

41. A radiological imaging method comprising:
2 radiation sources with imaging directions orthogonal to each other, one frontal radiation source and one lateral radiation source, sliding vertically so as to perform vertical scanning of a standing patient along a vertical scanning direction,
2 radiation detectors which are respectively associated with said 2 radiations sources, one frontal radiation detector and one lateral radiation detector, sliding vertically so as to perform vertical scanning of a standing patient along said vertical scanning direction, at least said lateral radiation detector being a multi-energy counting detector,
wherein said radiological method comprises at least one operating mode in which:
a lateral mono-energy scout view is made by performing a preliminary vertical scanning of a standing patient along said vertical scanning direction by said lateral radiation source and by said lateral radiation detector,
said lateral scout view is processed to identify a patient thickness and a specific bone(s) localization at different positions along said vertical scanning direction within said lateral scout view,
a driving current intensity of at least said lateral radiation source is modulated along said vertical scanning direction, depending on identified patient thickness and on said identified specific bone(s) localization at different positions along said vertical scanning direction,
so that a lateral multi-energy image is made by performing a single vertical scanning of a standing patient along said vertical scanning direction by said lateral radiation source and by said lateral radiation detector,
with a driving current intensity modulation of said lateral radiation source, with no voltage intensity modulation of said lateral radiation source, depending on said patient thickness and depending on said identified specific bone(s) localization at different positions along said vertical scanning direction, which is performed automatically, so as to improve a compromise between:
the global radiation dose received by a patient during said vertical scanning,
and the local image contrasts of said identified specific bone(s) localization at different imaging positions along said vertical scanning direction, for, at least one or more, or preferably all, lateral images given by said lateral radiation detector,
so that said lateral radiation detector gives at least, after said single vertical scanning:
a first lateral image corresponding to a first portion of energy which is received by said lateral radiation detector and which is below a first given energy threshold, called low energy lateral image,
a second lateral image corresponding to a second portion of energy which is received by said lateral radiation detector and which is above a second given energy threshold, called high energy lateral image,
at least a combined lateral image corresponding to a combination of said first lateral image and said second lateral image.

42. A radiological imaging method comprising:
2 radiation sources with imaging directions orthogonal to each other, one frontal radiation source and one lateral radiation source, sliding vertically so as to perform vertical scanning of a standing patient along a vertical scanning direction,
2 radiation detectors which are respectively associated with said 2 radiations sources, one frontal radiation detector and one lateral radiation detector, sliding vertically so as to perform vertical scanning of a standing patient along said vertical scanning direction, said 2 radiation detectors being respectively 2 multi-energy counting detectors,
wherein said radiological method comprises at least one operating mode in which:
frontal and lateral mono-energy scout views are made by performing a preliminary vertical scanning of a standing patient along said vertical scanning direction by said frontal and lateral radiation sources and by said frontal and lateral radiation detectors,
said frontal and lateral scout views are processed to identify a patient thickness and a specific bone(s) localization at different positions along said vertical scanning direction within said frontal and lateral scout views,
driving current intensities of both said frontal and lateral radiation sources are modulated along said vertical scanning direction, depending on identified patient thickness and on said identified specific bone(s) localization at different positions along said vertical scanning direction,
so that a frontal multi-energy image is made by performing a single vertical scanning of a standing patient along said vertical scanning direction by said frontal radiation source and by said frontal radiation detector,
with a driving current intensity modulation of said frontal radiation source, with no voltage intensity modulation of said frontal radiation source, depending on said patient thickness and depending on said identified specific bone(s) localization at different positions along said vertical scanning direction, which is performed automatically, so as to improve a compromise between:
the global radiation dose received by a patient during said vertical scanning,
and the local image contrasts of said identified specific bone(s) localization at different imaging positions along said vertical scanning direction, for, at least one or more, or preferably all, frontal images given by said frontal radiation detector,
so that said frontal radiation detector gives at least, after said single vertical scanning:
a first frontal image corresponding to a first portion of energy which is received by said frontal radiation detector and which is below a first given energy threshold, called low energy frontal image,
a second frontal image corresponding to a second portion of energy which is received by said frontal radiation detector and which is above a second given energy threshold, called high energy frontal image,
at least a combined frontal image corresponding to a combination of said first frontal image and said second frontal image,
and so that a lateral multi-energy image is made by performing a vertical scanning of a standing patient along said vertical scanning direction by said lateral radiation source and by said lateral radiation detector,
with a driving current intensity modulation of said lateral radiation source, with no voltage intensity modulation of said lateral radiation source, depending on said patient thickness and depending on said identified specific bone(s) localization at different positions along said vertical scanning direction, which is performed automatically, so as to improve a compromise between:
the global radiation dose received by a patient during said vertical scanning,
and the local image contrasts of said identified specific bone(s) localization at different imaging positions along said vertical scanning direction, for, at least one or more, or preferably all, lateral images given by said lateral radiation detector,
so that said lateral radiation detector gives at least:
a first lateral image corresponding to a first portion of energy which is received by said lateral radiation detector and which is below a first given energy threshold, called low energy lateral image,
a second lateral image corresponding to a second portion of energy which is received by said lateral radiation detector and which is above a second given energy threshold, called high energy lateral image,
at least a combined lateral image corresponding to a combination of said first lateral image and said second lateral image,
both frontal and lateral multi-energy images being made during same vertical scanning.

43. A radiological imaging method comprising:
2 radiation sources with imaging directions orthogonal to each other, one frontal radiation source and one lateral radiation source, sliding vertically so as to perform vertical scanning of a standing patient along a vertical scanning direction,
2 radiation detectors which are respectively associated with said 2 radiations sources, one frontal radiation detector and one lateral radiation detector, sliding vertically so as to perform vertical scanning of a standing patient along said vertical scanning direction, said 2 radiation detectors being respectively 2 multi-energy counting detectors,
wherein said radiological method comprises at least one operating mode in which:
a frontal multi-energy scout view is made by performing a preliminary vertical scanning of a standing patient along said vertical scanning direction by said frontal radiation source and by said frontal radiation detector, so that said frontal radiation detector gives at least:
a first frontal scout view corresponding to a first portion of energy which is received by said frontal radiation detector and which is below a first given energy threshold, called low energy frontal scout view,
a second frontal scout view corresponding to a second portion of energy which is received by said frontal radiation detector and which is above a second given energy threshold, called high energy frontal scout view,
said first frontal scout view and said second frontal scout view are combined and processed so as to evaluate:
at least a patient bone thickness,
at least a patient soft tissue thickness,
a patient specific bone localization at different imaging positions along said vertical scanning direction,
driving current intensity of said frontal radiation source is modulated along said vertical scanning direction, depending on said patient bone thickness, on said patient soft tissue thickness, and on said identified specific bone(s) localization at different positions along said vertical scanning direction,
so that a frontal multi-energy image is made by performing a single vertical scanning of a standing patient along said vertical scanning direction by said frontal radiation source and by said frontal radiation detector,
with a driving current intensity modulation of said frontal radiation source, with no voltage intensity modulation of said frontal radiation source, depending on said patient bone thickness, on said patient soft tissue thickness, and depending on said identified specific bone(s) localization at different positions along said vertical scanning direction, which is performed automatically, so as to improve a compromise between:
the global radiation dose received by a patient during said vertical scanning,
and the local image contrasts of said identified specific bone(s) localization at different imaging positions along said vertical scanning direction, for, at least one or more, or preferably all, frontal images given by said frontal radiation detector,
so that said frontal radiation detector gives at least, after said single vertical scanning:
a first frontal image corresponding to a first portion of energy which is received by said frontal radiation detector and which is below a first given energy threshold, called low energy frontal image,
a second frontal image corresponding to a second portion of energy which is received by said frontal radiation detector and which is above a second given energy threshold, called high energy frontal image,
at least a combined frontal image corresponding to a combination of said first frontal image and said second frontal image.

44. A radiological imaging method comprising:
2 radiation sources with imaging directions orthogonal to each other, one frontal radiation source and one lateral radiation source, sliding vertically so as to perform vertical scanning of a standing patient along a vertical scanning direction,
2 radiation detectors which are respectively associated with said 2 radiations sources, one frontal radiation detector and one lateral radiation detector, sliding vertically so as to perform vertical scanning of a standing patient along said vertical scanning direction, said 2 radiation detectors being respectively 2 multi-energy counting detectors,
wherein said radiological method comprises at least one operating mode in which:
a lateral multi-energy scout view is made by performing a preliminary vertical scanning of a standing patient along said vertical scanning direction by said lateral radiation source and by said lateral radiation detector, so that said lateral radiation detector gives at least:
a first lateral scout view corresponding to a first portion of energy which is received by said lateral radiation detector and which is below a first given energy threshold, called low energy lateral scout view,
a second lateral scout view corresponding to a second portion of energy which is received by said lateral radiation detector and which is above a second given energy threshold, called high energy lateral scout view,
said first lateral scout view and said second lateral scout view are combined and processed so as to evaluate:
at least a patient bone thickness,
at least a patient soft tissue thickness, and
a patient specific bone localization at different imaging positions along said vertical scanning direction,
driving current intensity of said lateral radiation source is modulated along said vertical scanning direction, depending on said patient bone thickness, on said patient soft tissue thickness, and on said identified specific bone(s) localization at different positions along said vertical scanning direction,
so that a lateral multi-energy image is made by performing a single vertical scanning of a standing patient along said vertical scanning direction by said lateral radiation source and by said lateral radiation detector,
with a driving current intensity modulation of said lateral radiation source, with no voltage intensity modulation of said lateral radiation source, depending on said patient bone thickness, on said patient soft tissue thickness, and depending on said identified specific bone(s) localization at different positions along said vertical scanning direction, which is performed automatically, so as to improve a compromise between:
the global radiation dose received by a patient during said vertical scanning,
and the local image contrasts of said identified specific bone(s) localization at different imaging positions along said vertical scanning direction, for, at least one or more, or preferably all, lateral images given by said lateral radiation detector,
so that said lateral radiation detector gives at least, after said single vertical scanning:
a first lateral image corresponding to a first portion of energy which is received by said lateral radiation detector and which is below a first given energy threshold, called low energy lateral image,
a second lateral image corresponding to a second portion of energy which is received by said lateral radiation detector and which is above a second given energy threshold, called high energy lateral image, and
at least a combined lateral image corresponding to a combination of said first lateral image and said second lateral image.

45. A radiological imaging method comprising:
2 radiation sources with imaging directions orthogonal to each other, one frontal radiation source and one lateral radiation source, sliding vertically so as to perform vertical scanning of a standing patient along a vertical scanning direction,
2 radiation detectors which are respectively associated with said 2 radiations sources, one frontal radiation detector and one lateral radiation detector, sliding vertically so as to perform vertical scanning of a standing patient along said vertical scanning direction, said 2 radiation detectors being respectively 2 multi-energy counting detectors,
wherein said radiological method comprises at least one operating mode in which:
frontal and lateral multi-energy scout views are made by performing a preliminary vertical scanning of a standing patient along said vertical scanning direction by said frontal and lateral radiation sources and by said frontal and lateral radiation detectors, so that said frontal and lateral radiation detectors give at least:
a first frontal scout view corresponding to a first portion of energy which is received by said frontal radiation detector and which is below a first given energy threshold, called low energy frontal scout view,
a second frontal scout view corresponding to a second portion of energy which is received by said frontal radiation detector and which is above a second given energy threshold, called high energy frontal scout view,
a first lateral scout view corresponding to a first portion of energy which is received by said lateral radiation detector and which is below a first given energy threshold, called low energy lateral scout view,
a second lateral scout view corresponding to a second portion of energy which is received by said lateral radiation detector and which is above a second given energy threshold, called high energy lateral scout view,
said first frontal and lateral scout views and said second frontal and lateral scout views are combined and processed so as to evaluate:
at least a patient bone thickness,
at least a patient soft tissue thickness,
a patient specific bone localization at different imaging positions along said vertical scanning direction,
driving current intensities of both said frontal and lateral radiation sources are modulated along said vertical scanning direction, depending on said patient bone thickness, on said patient soft tissue thickness, and on said identified specific bone(s) localization at different positions along said vertical scanning direction,
so that a frontal multi-energy image is made by performing a single vertical scanning of a standing patient along said vertical scanning direction by said frontal radiation source and by said frontal radiation detector,
with a driving current intensity modulation of said frontal radiation source, with no voltage intensity modulation of said frontal radiation source, depending on said patient bone thickness, on said patient soft tissue thickness, and depending on said identified specific bone(s) localization at different positions along said vertical scanning direction, which is performed automatically, so as to improve a compromise between:
the global radiation dose received by a patient during said vertical scanning,
and the local image contrasts of said identified specific bone(s) localization at different imaging positions along said vertical scanning direction, for, at least one or more, or preferably all, frontal images given by said frontal radiation detector,
so that said frontal radiation detector gives at least, after said single vertical scanning:
a first frontal image corresponding to a first portion of energy which is received by said frontal radiation detector and which is below a first given energy threshold, called low energy frontal image,
a second frontal image corresponding to a second portion of energy which is received by said frontal radiation detector and which is above a second given energy threshold, called high energy frontal image, and
at least a combined frontal image corresponding to a combination of said first frontal image and said second frontal image,
and so that a lateral multi-energy image is made by performing a vertical scanning of a standing patient along said vertical scanning direction by said lateral radiation source and by said lateral radiation detector,
with a driving current intensity modulation of said lateral radiation source, with no voltage intensity modulation of said lateral radiation source, depending on said patient bone thickness, on said patient soft tissue thickness, and depending on said identified specific bone(s) localization at different positions along said vertical scanning direction, which is performed automatically, so as to improve a compromise between:
the global radiation dose received by a patient during said vertical scanning,
and the local image contrasts of said identified specific bone(s) localization at different imaging positions along said vertical scanning direction, for, at least one or more, or preferably all, lateral images given by said lateral radiation detector,
so that said lateral radiation detector gives at least:
a first lateral image corresponding to a first portion of energy which is received by said lateral radiation detector and which is below a first given energy threshold, called low energy lateral image,
a second lateral image corresponding to a second portion of energy which is received by said lateral radiation detector and which is above a second given energy threshold, called high energy lateral image,
at least a combined lateral image corresponding to a combination of said first lateral image and said second lateral image,
both frontal and lateral multi-energy images being made during same vertical scanning.

46. The radiological imaging method according to claim 43, wherein:
said first and second frontal scout views are processed to a multi-material decomposition with at least two material thickness vertical profiles,
preferably, either a bi-material decomposition between Al and PMMA, or a bi-material decomposition between HA and H₂O,
and/or said first and second lateral scout views are processed to a multi-material decomposition with at least two material thickness vertical vectors,
preferably, either a bi-material decomposition between Al and PMMA, or a bi-material decomposition between HA and H₂O.

47. The radiological imaging method according to claim 40, wherein:
said frontal mono-energy scout view is made by performing a single preliminary vertical scanning of a standing patient along said vertical scanning direction by said frontal radiation source and by said frontal radiation detector,
said lateral mono-energy scout view is made by performing a single preliminary vertical scanning of a standing patient along said vertical scanning direction by said lateral radiation source and by said lateral radiation detector, and
both said frontal mono-energy scout view and said lateral mono-energy scout view being made during same single vertical scanning.

48. The radiological imaging method according to claim 40, wherein said first given energy threshold is equal or less than said second given energy threshold, preferably equal to said second given energy threshold,
and preferably wherein:
said first given energy threshold is equal to said second given energy threshold,
said frontal and/or lateral multi-energy images are made (3) so that said frontal and/or lateral radiation detectors (122, 124) first give:
said first frontal image,
a third frontal image corresponding to the whole energy which is received by said frontal radiation detector (122), called total energy frontal image,
said second frontal image being obtained by a subtracting said first frontal image from said third frontal image,
and/or said first lateral image,
and/or a third lateral image corresponding to the whole energy which is received by said lateral radiation detector (124), called total energy lateral image,
said second lateral image being obtained by a subtracting said first lateral image from said third lateral image.

49. The radiological imaging method according to claim 40, wherein:
said frontal image is made by performing a vertical scanning of a standing patient along said vertical scanning direction by said frontal radiation source and by said frontal radiation detector, with:
said modulation of a driving current intensity of at least said frontal radiation source along said vertical scanning direction, depending on said patient thickness and on said specific bone(s) localization at different positions along said vertical scanning direction,
said lateral image is made by performing a vertical scanning of a standing patient along said vertical scanning direction by said lateral radiation source and by said lateral radiation detector, with:
said modulation of a driving current intensity of at least said lateral radiation source along said vertical scanning direction, depending on said patient thickness and on said specific bone(s) localization at different positions along said vertical scanning direction, and
both said frontal image and said lateral image being made during same vertical scanning.

50. The radiological imaging method according to claim 40, wherein:
said driving current intensity modulation(s) of said frontal and/or lateral radiation source(s), with no voltage intensity modulation of said frontal and/or lateral radiation source(s), is performed automatically, so as to improve a compromise between:
lowering the global radiation dose received by a patient during said vertical scanning,
and not degrading under a given contrast threshold the local image contrasts of said identified specific bone(s) localization at different imaging positions along said vertical scanning direction, for all or part of patient thicknesses along said vertical scanning direction, for the frontal and/or lateral image(s),
and/or wherein:
said driving current intensity modulation(s) of said frontal and/or lateral radiation source(s), with no voltage intensity modulation of said frontal and/or lateral radiation source(s), is performed automatically, so as to improve a compromise between:
lowering the global radiation dose received by a patient during said vertical scanning,
and improving the contrast to noise ratio or the ratio between contrast to noise ratio and square root of said global radiation dose of said identified specific bone(s) localization at different imaging positions along said vertical scanning direction, with respect to local image contrasts of said identified specific bone(s) localization at different imaging positions along said vertical scanning direction with same global radiation dose but without any driving current intensity modulation, for all or part of patient thicknesses along said vertical scanning direction, for the frontal and/or lateral image(s).

51. The radiological imaging method according to claim 40, wherein:
said frontal multi-energy image acquisition is performed with at least 2 energy bins, or with at least 3 energy bins, or with at least 6 energy bins,
and/or at most 20 energy bins, or at most 15 energy bins, or at most 10 energy bins,
and/or said lateral multi-energy image acquisition is performed with at least 2 energy bins, or with at least 3 energy bins, or with 6 energy bins,
and/or at most 20 energy bins, or at most 15 energy bins, or at most 10 energy bins.

52. The radiological imaging method according to claim 40, wherein said driving current intensity modulation of said frontal and/or lateral radiation source(s) is performed also so as to reach a value of signal to noise ratio which is constant and common to most of said imaging positions along said vertical scanning direction, preferably to all said imaging positions along said vertical scanning direction, for said frontal image and/or for said lateral image, but which can take two different values respectively for frontal image and for lateral image.

53. The radiological imaging method according to claim 52, wherein, for each of said frontal and/or lateral images, said signal to noise ratio value is constant and predetermined for each different patient organ to be imaged,
and/or wherein:
for a frontal image of a patient spine, said standard signal to noise ratio value corresponds to a number of X-ray photons received per detector pixel comprised between 50 and 70, the radiological imaging method operator preferably having the possibility to deviate, via a manual command, from this standard value by at least −25% or +100%, more preferably by at least −50% or +200%,
and/or for a lateral image of a patient spine, said standard signal to noise ratio value corresponds to a number of X-ray photons received per detector pixel comprised between 20 and 40, the radiological imaging method operator preferably having the possibility to deviate, via a manual command, from this standard value by at least −25% or +100%, more preferably by at least −50% or +200%.

54. The radiological imaging method according to claim 40, wherein said current intensity modulation is maximized so as to also maximize said vertical scanning speed at a constant value.

55. The radiological imaging method according to claim 40, wherein said current intensity modulation(s) rate do(es) not go beyond a predetermined threshold of 5 mA per millisecond, or do(es) not go beyond a predetermined threshold of 2 mA per millisecond, or do(es) not go beyond a predetermined threshold of 1 mA per millisecond,
and/or wherein said current intensity modulation(s) at least range(s) from 20 mA to 300 mA, and preferably from 10 mA to 400 mA,
and/or wherein said vertical scanning speed value at least range(s) from 8 cm/second to 20 cm/second, and preferably from 0.4 cm/second to 35 cm/second.

56. The radiological imaging method according to claim 40, wherein each of said frontal and/or lateral scout view(s) is made by performing a preliminary vertical scanning of a standing patient along a vertical scanning direction with a reduced global radiation dose as compared to each of said frontal and lateral images, before making each of said frontal and lateral images, and preferably wherein said reduced global radiation is less than 10% of said global radiation dose, more preferably less than 5% of said global radiation dose.

57. The radiological imaging method according to claim 40, wherein pixels in said scout view are gathered together, preferably by zones of N×N pixels, more preferably by zones ranging from 2×2 pixels to of 10×10 pixels, to make imaged zones.

58. The radiological imaging method according to claim 40, wherein said images or said imaged zones are processed to identify salient points which in turn are used to compute said thickness profile and to identify said specific bone(s) localization of a standing patient along said vertical scanning direction.

59. The radiological imaging method according to claim 40, wherein said 2 radiation detectors are respectively associated with said 2 radiations sources, said 2 radiation detectors being 2 Photon Counting Detectors (PCD) each being associated to an automatic image processing function automatically balancing image density whatever radiation dose received on the sensitive surface of said radiation detector to homogenize responses of said detectors,
and/or wherein said 2 radiation detectors are respectively associated with said 2 radiations sources, said 2 radiation detectors being 2 multi-energy counting detectors, preferably being 2 Energy Resolved Photon Counting Detectors (ERPCD).

60. The radiological imaging method according to claim 40, wherein the voltage intensity of said frontal radiation source is more than 90 kVp, or more preferably more than 100 kVp.

61. The radiological imaging method according to claim 40, wherein said second energy threshold is chosen so as to improve image contrast more for lower patient thicknesses regions along vertical direction than for higher patient thicknesses regions along vertical direction, preferably said second energy threshold being chosen between 50 keV and 90 keV, preferably between 60 keV and 80 keV, more preferably said second energy threshold being chosen at 70 keV.

62. The radiological imaging method according to claim 40, wherein:
said second frontal image includes information which allows for assessing a patient bone density, and said second lateral image includes information which allows for assessing a patient bone density,
and/or said combined frontal image presents local image contrasts of said identified specific bone(s) localization at different imaging positions along said vertical scanning direction which are sufficient to perform a diagnostic on a patient, and said combined lateral image presents local image contrasts of said identified specific bone(s) localization at different imaging positions along said vertical scanning direction which are sufficient to perform a diagnostic on a patient.

63. The radiological imaging method according to claim 43, wherein said first given energy threshold is equal or less than said second given energy threshold, preferably equal to said second given energy threshold,
and preferably wherein:
said first given energy threshold is equal to said second given energy threshold,
said frontal and/or lateral multi-energy images are made (3) so that said frontal and/or lateral radiation detectors (122, 124) first give:
said first frontal image,
a third frontal image corresponding to the whole energy which is received by said frontal radiation detector (122), called total energy frontal image,
said second frontal image being obtained by a subtracting said first frontal image from said third frontal image,
and/or said first lateral image,
and/or a third lateral image corresponding to the whole energy which is received by said lateral radiation detector (124), called total energy lateral image,
said second lateral image being obtained by a subtracting said first lateral image from said third lateral image.

64. The radiological imaging method according to claim 43, wherein:
said frontal image is made by performing a vertical scanning of a standing patient along said vertical scanning direction by said frontal radiation source and by said frontal radiation detector, with:
said modulation of a driving current intensity of at least said frontal radiation source along said vertical scanning direction, depending on said patient thickness and on said specific bone(s) localization at different positions along said vertical scanning direction,
said lateral image is made by performing a vertical scanning of a standing patient along said vertical scanning direction by said lateral radiation source and by said lateral radiation detector, with:
said modulation of a driving current intensity of at least said lateral radiation source along said vertical scanning direction, depending on said patient thickness and on said specific bone(s) localization at different positions along said vertical scanning direction, and
both said frontal image and said lateral image being made (3) during same vertical scanning.

65. The radiological imaging method according to claim 43, wherein:
said driving current intensity modulation(s) of said frontal and/or lateral radiation source(s), with no voltage intensity modulation of said frontal and/or lateral radiation source(s), is performed automatically, so as to improve a compromise between:
lowering the global radiation dose received by a patient during said vertical scanning,
and not degrading under a given contrast threshold the local image contrasts of said identified specific bone(s) localization at different imaging positions along said vertical scanning direction, for all or part of patient thicknesses along said vertical scanning direction, for the frontal and/or lateral image(s),
and/or wherein:
said driving current intensity modulation(s) of said frontal and/or lateral radiation source(s), with no voltage intensity modulation of said frontal and/or lateral radiation source(s), is performed automatically, so as to improve a compromise between:
lowering the global radiation dose received by a patient during said vertical scanning,
and improving the contrast to noise ratio or the ratio between contrast to noise ratio and square root of said global radiation dose of said identified specific bone(s) localization at different imaging positions along said vertical scanning direction, with respect to local image contrasts of said identified specific bone(s) localization at different imaging positions along said vertical scanning direction with same global radiation dose but without any driving current intensity modulation, for all or part of patient thicknesses along said vertical scanning direction, for the frontal and/or lateral image(s).

66. The radiological imaging method according to claim 43, wherein:
said frontal multi-energy image acquisition is performed with at least 2 energy bins, or with at least 3 energy bins, or with at least 6 energy bins,
and/or at most 20 energy bins, or at most 15 energy bins, or at most 10 energy bins,
and/or said lateral multi-energy image acquisition is performed with at least 2 energy bins, or with at least 3 energy bins, or with 6 energy bins,
and/or at most 20 energy bins, or at most 15 energy bins, or at most 10 energy bins.

67. The radiological imaging method according to claim 43, wherein said driving current intensity modulation of said frontal and/or lateral radiation source(s) is performed also so as to reach a value of signal to noise ratio which is constant and common to most of said imaging positions along said vertical scanning direction, preferably to all said imaging positions along said vertical scanning direction, for said frontal image and/or for said lateral image, but which can take two different values respectively for frontal image and for lateral image.

68. The radiological imaging method according to claim 67, wherein, for each of said frontal and/or lateral images, said signal to noise ratio value is constant and predetermined for each different patient organ to be imaged,
and/or wherein:
for a frontal image of a patient spine, said standard signal to noise ratio value corresponds to a number of X-ray photons received per detector pixel comprised between 50 and 70, the radiological imaging method operator preferably having the possibility to deviate, via a manual command, from this standard value by at least −25% or +100%, more preferably by at least −50% or +200%,
and/or for a lateral image of a patient spine, said standard signal to noise ratio value corresponds to a number of X-ray photons received per detector pixel comprised between 20 and 40, the radiological imaging method operator preferably having the possibility to deviate, via a manual command, from this standard value by at least −25% or +100%, more preferably by at least −50% or +200%.

69. The radiological imaging method according to claim 43, wherein said current intensity modulation is maximized so as to also maximize said vertical scanning speed at a constant value.

70. The radiological imaging method according to claim 43, wherein said current intensity modulation(s) rate do(es) not go beyond a predetermined threshold of 5 mA per millisecond, or do(es) not go beyond a predetermined threshold of 2 mA per millisecond, or do(es) not go beyond a predetermined threshold of 1 mA per millisecond,
and/or wherein said current intensity modulation(s) at least range(s) from 20 mA to 300 mA, and preferably from 10 mA to 400 mA,
and/or wherein said vertical scanning speed value at least range(s) from 8 cm/second to 20 cm/second, and preferably from 0.4 cm/second to 35 cm/second.

71. The radiological imaging method according to claim 43, wherein each of said frontal and/or lateral scout view(s) is made by performing a preliminary vertical scanning of a standing patient along a vertical scanning direction with a reduced global radiation dose as compared to each of said frontal and lateral images, before making each of said frontal and lateral images, and preferably wherein said reduced global radiation is less than 10% of said global radiation dose, more preferably less than 5% of said global radiation dose.

72. The radiological imaging method according to claim 43, wherein pixels in said scout view are gathered together, preferably by zones of N×N pixels, more preferably by zones ranging from 2×2 pixels to of 10×10 pixels, to make imaged zones.

73. The radiological imaging method according to claim 43, wherein said images or said imaged zones are processed to identify salient points which in turn are used to compute said thickness profile and to identify said specific bone(s) localization of a standing patient along said vertical scanning direction.

74. The radiological imaging method according to claim 43, wherein said 2 radiation detectors are respectively associated with said 2 radiations sources, said 2 radiation detectors being 2 Photon Counting Detectors (PCD) each being associated to an automatic image processing function automatically balancing image density whatever radiation dose received on the sensitive surface of said radiation detector to homogenize responses of said detectors,
and/or wherein said 2 radiation detectors are respectively associated with said 2 radiations sources, said 2 radiation detectors being 2 multi-energy counting detectors, preferably being 2 Energy Resolved Photon Counting Detectors (ERPCD).

75. The radiological imaging method according to claim 43, wherein the voltage intensity of said frontal radiation source is more than 90 kVp, or more preferably more than 100 kVp.

76. The radiological imaging method according to claim 43, wherein said second energy threshold is chosen so as to improve image contrast more for lower patient thicknesses regions along vertical direction than for higher patient thicknesses regions along vertical direction, preferably said second energy threshold being chosen between 50 keV and 90 keV, preferably between 60 keV and 80 keV, more preferably said second energy threshold being chosen at 70 keV.

77. The radiological imaging method according to claim 43, wherein:
said second frontal image includes information which allows for assessing 7 a patient bone density, and said second lateral image includes information which allows for assessing 7a patient bone density,
and/or said combined frontal image presents local image contrasts of said identified specific bone(s) localization at different imaging positions along said vertical scanning direction which are sufficient to perform 7a diagnostic on a patient, and said combined lateral image presents local image contrasts of said identified specific bone(s) localization at different imaging positions along said vertical scanning direction which are sufficient to perform (5) a diagnostic on a patient.
